(19) United States
(12) Patent Application Publication
(10) Pub. No.: US 2024/0408279 A1
(43) Pub. Date: Dec. 12, 2024

(54) SUPRAMOLECULAR-COVALENT COMPOSITE SLURRY FOR CARTILAGE REPAIR

(71) Applicant: Northwestern University, Evanston, IL (US)

(72) Inventors: Jacob A. LEWIS, Evanston, IL (US); Mark Trosper MCCLENDON, Evanston, IL (US); Samuel I. STUPP, Evanston, IL (US); Nicholas A. SATHER, Evanston, IL (US)

(21) Appl. No.: 18/697,681

(22) PCT Filed: Sep. 30, 2022
(86) PCT No.: PCT/US2022/077368

§ 371 Date: Apr. 1, 2024

Related U.S. Application Data
(60) Provisional application No. 63/251,328, filed on Oct. 1, 2021.

(51) Int. Cl. A61L 27/54; A61L 27/48; A61L 27/52

(52) U.S. Cl. CPC A61L 27/54; A61L 27/48; A61L 27/52; A61L 2300/236; A61L 2300/414; A61L 2400/12; A61L 2430/06

(57) ABSTRACT

Provided herein are compositions comprising a composite of peptide amphiphile nanofibers and hyaluronic acid (HA) particles, and methods of preparation and use thereof, such as for repair of a cartilage or osteochondral defects.

### CROSS-REFERENCE TO RELATED APPLICATIONS

This application claims the benefit of U.S. Provisional Patent Application No. 63/251,328, filed on Oct. 1, 2021, which is incorporated by reference herein.

### FIELD

Provided herein are compositions comprising a composite of peptide amphiphile nanofibers and hyaluronic acid (HA) particles, and methods of preparation and use thereof, such as for repair of a cartilage or osteochondral defects.

### BACKGROUND

Cartilage and osteochondral defects affect over 17 million Americans. Current cartilage repair techniques, such as microfracture, result in inferior fibrocartilage formation instead of healthy tissue. Previous work showed improved repair through implanting growth factor-binding nanofibers in rabbit trochlear defects; however, the material was unable to withstand the mechanical shear in large-animal joints.

### SUMMARY

Provided herein are compositions comprising a composite of peptide amphiphile nanofibers and hyaluronic acid (HA) particles, and methods of preparation and use thereof, such as for repair of a cartilage or osteochondral defects.

In some embodiments, provided herein are composite materials comprising: (1) peptide amphiphile nanofibers, and (2) hyaluronic acid (HA) particles. In some embodiments, the composite material comprises a paste-like consistency. In some embodiments, the composite material further comprising one or more growth factors.

In some embodiments, the HA particles are microparticles or nanoparticles. In some embodiments, the HA particles comprise crosslinked HA. In some embodiments, the HA particles comprise L-lysine methyl ester and 1-Ethyl-3-(3-dimethylaminopropyl) carbodiimide (EDC) crosslinked HA. In some embodiments, the HA particles are 5-50% crosslinked (e.g., 5%, 10%, 15%, 20%, 25%, 30%, 35%, 40%, 45%, 50%, or ranges therebetween). In some embodiments, are about 21% crosslinked. In some embodiments, the composite material comprises 2-10% HA (e.g., 2%, 3%, 4%, 5%, 6%, 7%, 8%, 9%, 10%, or ranges therebetween). In some embodiments, the composite material comprises about 4% HA. In some embodiments, the HA particles comprise oxidized HA. In some embodiments, the HA particles comprise partially-oxidized HA (e.g., 1-99% (1%, 2%, 5%, 10%, 20%, 30%, 40%, 50%, 60%, 70%, 80%, 90%, 95%, 99%, or ranges therebetween) of D-glucuronic acid monomers of HA oxidized to display aldehydes). In some embodiments, the HA particles display aldehyde groups suitable for crosslinking to amine groups.

In some embodiments, the peptide amphiphile nanofibers are derived from a peptide amphiphile solution. In some embodiments, the peptide amphiphile nanofibers comprise: (i) a hydrophobic non-peptidic segment; (ii) a β-sheet-forming peptide segment; (iii) an acidic peptide segment; and (iv) a bioactive peptide. In some embodiments, the bioactive peptide binds a growth factor or other bioactive agent. In some embodiments, the bioactive peptide binds TGF-β1. In some embodiments, the hydrophobic non-peptidic segment of the bioactive peptide amphiphile comprises an acyl chain. In some embodiments, the acyl chain comprises C₆-C₂₀. In some embodiments, the acyl chain comprises C16. In some embodiments, the β-sheet-forming peptide segment of the bioactive peptide amphiphile comprises a combination of 2-6 V and A residues. In some embodiments, the β-sheet-forming peptide segment of the bioactive peptide amphiphile and the charged peptide amphiphile is selected from VVVAAA, AAAVVV, AAVV, VVAA, AA, VV, VA, or AV. In some embodiments, the acidic peptide segment of the bioactive peptide amphiphile comprises a combination of 1-4 Glu (E) and/or Asp (D) residues. In some embodiments, the acidic peptide segment comprises is selected from E, EE, EEE, D, DD, DDD, ED, DE, EDE, DED, EDD, and DEE. In some embodiments, a backbone PA of the bioactive peptide amphiphile selected from C16-AAEE, C16-AEAE, and 16-VVVAAAEEE.

In some embodiments, the PA nanofibers further comprise a diluent PA comprising: (i) a hydrophobic non-peptidic segment; (ii) a β-sheet-forming peptide segment; and (iii) a charged peptide segment. In some embodiments, the hydrophobic non-peptidic segment of the diluent peptide amphiphile comprises an acyl chain. In some embodiments, the acyl chain comprises C₆-C₂₀. In some embodiments, the acyl chain comprises C16. In some embodiments, the β-sheet-forming peptide segment of the diluent peptide amphiphile comprises a combination of 2-6 V and A residues. In some embodiments, the β-sheet-forming peptide segment of the diluent peptide amphiphile is selected from VVVAAA, AAAVVV, AAVV, VVAA, AA, VV, VA, or AV. In some embodiments, the acidic peptide segment of the diluent peptide amphiphile comprises a combination of 1-4 Glu (E) and/or Asp (D) residues. In some embodiments, the acidic peptide segment of the diluent peptide amphiphile is selected from E, EE, EEE, D, DD, DDD, ED, DE, EDE, DED, EDD, and DEE. In some embodiments, a backbone PA of the diluent peptide amphiphile selected from C16-AAEE, C16-AEAE, and 16-VVVAAAEEE.

In some embodiments, the PA nanofibers have a ratio of between 1:1 and 1:25 bioactive PA to diluent PA (e.g., 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, or ranges therebetween).

In some embodiments, provided herein are methods of promoting repair of a cartilage and/or osteochondral defect (e.g., in a subject) comprising administering the composite material herein to the cartilage and/or osteochondral defect. In some embodiments, the subject exhibits poor cartilage healing capacity. In some embodiments, the subject has an O'Driscoll score or modified O'Driscoll score of 20 or lower (e.g., 20, 19, 18, 17, 16, 15, 14, 13, 12, 11, 10, 9, 8, 7, 6, 5, or lower, or ranges therebetween (e.g., 15 or lower)).

In some embodiments, provided herein are methods of preparing a composite material herein comprising mixing a solution of peptide amphiphiles with HA particles in an aqueous solution.

In some embodiments, provided herein are systems comprising a composite material herein and a medical device for applying the composition to a cartilage and/or osteochondral defect in a subject. In some embodiments, the medical device is a syringe, catheter, or paste applicator.

In some embodiments, provided herein are systems comprising a composite material herein and a growth factor or other bioactive agent that bonds to the bioactive PA (e.g., TGF-β1).

### BRIEF DESCRIPTION OF THE DRAWINGS

FIG. 1A-H. Mechanical performance of supramolecular-covalent hybrid scaffold (a) chemical structure of pa molecules used. (b) storage modulus and loss modulus as a function of strain for gels comprising PA only, PA added to 4% unmodified HA, and PA added to 4% crosslinked HA particles with flow strain indicated by vertical lines (n=3). (c) Volume increase as a function of incubation time for PA only gels and PA/HA gels with unmodified or crosslinked HA; significance calculated relative to PA only gels (n=5). (d) Hyaluronidase catalyzed degradation of HA as a function of time for crosslinked HA alone, hybrid hydrogel of PA and unmodified HA, and hybrid hydrogel of PA and crosslinked HA determined using a carbazole assay (*, PA+HA vs. PA+X-link HA; #, PA+HA vs. X-link HA only; n=4). (e) Schematic showing preparation and application of PA/HA hybrid material. Photographs of (f) a 3-4 mm deep, 7 mm diameter shallow osteochondral defect made by drilling into the medial condyle of a sheep stifle, and (g) the defect with the PA/HA hybrid. H&E histological staining (h,j) and fluorescence imaging (i,k) produced by TAMRA-tagged PA filaments (red) in the PA/HA hybrid overlaying unstained sections for slung (h,i) and contralateral non-slung (j,k) stifles prepared from sections of the medial condyle obtained 7 d following implantation. Scale=1 mm. (*, p<0.05; **, p<0.01; ***, p<0.001).

FIG. 2A-K. Self-assembled morphology of scaffold and chondrogenesis of encapsulated cells. (a) SEM micrographs of gels formed by 2 wt % PA only, or the same concentration added to crosslinked HA for a final concentration of 2%, 4%, and 10%; scale=1 μm. (b) Schematic showing proposed arrangement of PA fibers (pink) and swelling HA particles (gray). (c) SAXS/MAXS/WAXS intensity as a function of the wave vector for PA only and PA/HA with d-spacings associated with observed peaks indicated. Immunocytochemistry confocal microscopy images showing an hMSC encapsulating (d) PA only gel and (e) PA/HA gel; nuclei stained with Hoechst (blue), actin stained with phalloidin (green), and PA tagged with TAMRA (red); scale=25 μm. (f) SEM micrograph an hMSC encapsulating PA/HA; scale=2 μm. (g) TGFβ-1 release from PA/HA gels as a function of time where gels were made either from filaments containing only the diluent PA or filaments containing the diluent and 10 mol % of the bioactive PA displaying the TGFβ-1 binding epitope (n=4). (h) sGAG content normalized to DNA concentration 4 weeks following encapsulation in PA only and PA/HA gels where gels were cultured without TGFβ-1, with TGFβ-1 supplemented media, or with TGFβ-1 added to the PA solution prior to cell encapsulation; significance relative to no TGFβ-1 control for each gel (n=5). (*, p<0.05; ***, p<0.001)

FIG. 3A-F. Osteochondral defect filling and scaffold integration at 4 weeks. Representative macroscopic appearance, Safranin O stained sections, and H&E stained sections (a,d) along with blinded ICRS scoring of macroscopic appearance (b,e) and of histological sections (c,f) of medial condyle defects treated with PA/HA hybrid material delivering TGFβ-1 and contralateral controls where TGFβ-1 was applied in saline solution (a-c) and of condyle defects treated with PA/HA hybrid material without growth factor and contralateral controls where only the saline vehicle was applied (d-f) 4 weeks following implantation (n=6). Scale=1 mm. (*, p<0.05; Mann-Whitney Test).

FIG. 4A-G. Repair of hyaline-like tissue at 12 weeks. Representative macroscopic appearance, Safranin O stained sections, H&E stained sections, and immunohistology staining of collagen II (a,d) with ICRS scoring of the macroscopic appearance (b,e) and O'Driscoll scoring of the histological sections (c,f) for medial condyle (a-c) and trochlear groove (d-f) osteochondral defects 12 weeks following implantation (n=13). (g) Representative macroscopic appearance, Safranin O stained sections, H&E stained sections, and immunohistology staining of collagen II for osteochondral condyle defects 24 weeks following implantation. Scale=1 mm. (*, p<0.05; Mann-Whitney Test).

FIG. 5A-C. LC-MS confirming purity and MS for (a) Diluent PA, (b) TGFβ-1 Binding PA, and (c) TAMRA-tagged PA.

FIG. 6. Conventional TEM imaging of heat treated PA filaments used to prepare PA/HA hybrid material used in surgery.

FIG. 7A-D. Preparation steps for crosslinked HA particles. (a) Viscous HA solution is mixed with crosslinker solution and poured into a flat dish to form (b) a film. (c) The film is rehydrated and washed to remove excess crosslinker and coupling agent and then shredded in a blender to form (d) a powder following lyophilization.

FIG. 8A-B. (a) Storage modulus and loss modulus as a function of strain for 2 wt % PA only samples and PA/HA samples comprising 2% PA and 2%, 4%, or 10% crosslinked HA (n=3). (b) Volume increase after 24 h in water for 2% PA only gels, 2% PA and 4% unmodified, 2% PA and 2% crosslinked HA, 2% PA and 4% crosslinked HA, and 2% PA and 10% crosslinked HA (*, p<0.05; **, p<0.01; ***, p<0.001; n=5).

FIG. 9A-F. Photographs of (a) a 3-4 mm deep, 7 mm diameter shallow osteochondral defect made by drilling into the trochlear groove of a sheep stifle, and (b) the defect filled with the PA/HA hybrid. H&E histological staining prepared from sections of the femoral trochlea obtained 7 d following implantation from (c) a slung stifle and (d) a contralateral non-slung stifle. Fluorescence imaging produced by TAMRA-tagged PA filaments (red) in the PA/HA hybrid overlaying unstained sections for the (e) slung and (f) non-slung stifles. Scale=1 mm.

FIG. 10A-B. SEM micrographs of (a) crosslinked HA particles alone, (b) 1% PA solution added to 4% crosslinked HA particles. Scale=1 μm.

FIG. 11. SAXS pattern produced by a 2 wt % PA solution (blue) fit to a core-shell cylinder model using SASView software (orange), with fitting parameters used to produce the displayed model.

FIG. 12. 2% PA solution added to 2% unmodified HA, 2% PA added to 4% unmodified HA, and 2% PA added to 10% unmodified HA. Scale=1 μm.

FIG. 13A-C. SAXS pattern for gels produced by adding (a) 2 wt % PA solutions, (b) 1 wt % PA solutions, or (c) water without PA to crosslinked HA or unmodified HA at concentration of 2%, 4%, and 10%.

FIG. 14. SEM micrograph of diluent PA only combined 4% crosslinked HA particles.

FIG. 15: Associated d-spacing for Bragg peak produced by PA/HA hybrid material as a function of combined PA and HA material concentration. Samples were prepared by serially diluting the PA solution and followed by addition to HA particles (blue) or by mixing the PA and HA solutions and then diluting (red).

FIG. 16A-B. (a) Quantification of fraction live cells as a function of encapsulating material. (b) Representative live/dead staining for cells encapsulated in PA gels or PA/HA gels cultured for 24 h. Live cells stained with calcein (green) and dead cells stained with propidium iodide (red).

FIG. 17A-B. (a) Representative macroscopic appearance, safranin O stained sections, and H&E stained sections of trochlear defects treated with PA/HA hybrid material delivering TGFβ-1 and contralateral controls where TGFβ-1 was applied in saline solution and of (b) condyle defects treated with PA/HA hybrid material without growth factor and contralateral controls where only the saline vehicle was applied 4 weeks following implantation. Scale=1 mm.

FIG. 18A-C. Macroscopic appearance and histological staining of (a) medial condyle and (b) trochlear groove defects treated with PA/HA hybrid material without growth factor and contralateral controls where only the saline vehicle was applied. (c) Comparison of median macroscopic scores and median histology scores with number of animals in each group and p-value calculated using a non-parametric Man-Whitney test. Scale=1 mm.

FIG. 19. Synovial biomarker concentration as a function of treatment at 0 d (before surgery) and 7 d post-surgery for interleuken 1α (IL-1α), interleuken 6 (IL-6), interleuken 8 (IL-8), interleuken 10 (IL-10), macrophage inflammatory protein 1α (MIP-1α), interleuken 36 receptor agonist (IL-36-RA), interferon gamma-induced protein 10 (IP-10), monocyte chemoattractant protein 1 (MCP1), tumor necrosis factor α (TNFα), and vascular endothelial growth factor (VEGF) detected using a MILLIPLEX Bovine Cytokine/Chemokine panel (n=13). (*, p<0.05; **, p<0.01; ***, p<0.001; significance calculated by Bonferroni pair-wise post-hoc test for 7 d relative to 0 d concentration).

FIG. 20. Mean histological scores by category for condyle and trochlea defects for the 12-week study in the (+) TGF group. n=13. 2-way ANOVA treatment effect: condyle, ns; trochlea, **. (*, p<0.05; **, p<0.01).

FIG. 21. Orthogonal crosslinking chemistry to enhance implant mechanics and retention in osteochondral defect. (A) Partially oxidized HA to produce aldehyde groups to be used for crosslinking to amine groups on PA nanofibers and on native cartilage and bone. (B) Strain sweeps of hybrid composites showing enhanced shear resistance in oxidized-HA/PA gels compared to HA/PA gels.

FIG. 22A-C. Histological analysis of sheep cartilage repair at 6 months post-implantation. (A) Representative H&E stained sections, Safranin O stained sections, and collagen II immunohistology stained sections for medial condyle osteochondral defects in the same sheep treated with 1 μg/mL TGFβ-1 delivered in saline (control) and in the PA/HA slurry (treatment). (B) Modified O'Driscoll scoring of the histological sections (*, p≤0.05; 1-tailed paired t-test). (C) Histological scores plotted with lines connecting the control and treatment scores for each animal. Animals with inherently poor healing (control side scores below 15) are shown in red.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine(S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine(S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO: Z (e.g., 100 amino acids) may have up to X substitutions (e.g., 10) relative to SEQ ID NO: Z, and may therefore also be expressed as “having X (e.g., 10) or fewer substitutions relative to SEQ ID NO:Z.”

As used herein, the term “nanofiber” refers to an elongated or threadlike filament (e.g., having a significantly greater length dimension that width or diameter) with a diameter typically less than 100 nanometers.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, marcomolecules, etc.) and the multicomponent assemblies, complexes, systems, and/or fibers that form as a result.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a non-peptide lipophilic (hydrophobic) segment, a structural peptide segment and/or charged peptide segment (often both), and optionally a bioactive segment (e.g., linker segment, bioactive segment, etc.). The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptide segment (e.g., comprising an acyl group of six or more carbons), (2) a structural peptide segment (e.g., β-sheet forming); (3) a charged peptide segment, and (4) a bioactive segment (e.g., linker segment).

As used herein and in the appended claims, the term “lipophilic moiety” or “hydrophobic moiety” refers to the moiety (e.g., an acyl, ether, sulfonamide, or phosphodiestermoiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and aggregate (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)— where n=2-25. In some embodiments, a linear acyl chain is the lipophilic group (saturated or unsaturated carbons), palmitic acid. However, other lipophilic groups may be used in place of the acyl chain such as steroids, phospholipids and fluorocarbons.

As used herein, the term “structural peptide” refers to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural segments of adjacent structural segments. In some embodiments, nanofibers of peptide amphiphiles having structural peptide segments display linear or 2D structure when examined by microscopy and/or α-helix and/or β-sheet character when examined by circular dichroism (CD).

As used herein, the term “beta (B)-sheet-forming peptide segment” refers to a structural peptide segment that has a propensity to display β-sheet-like character (e.g., when analyzed by CD). In some embodiments, amino acids in a beta (B)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the terms “carboxy-rich peptide segment,” “acidic peptide segment,” and “negatively-charged peptide segment” refer to a peptide sequence of a peptide amphiphile that comprises one or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues, or peptidomimetics with acidic side chains could be used, as will be evident to one ordinarily skilled in the art. There may be from about 2 to about 7 amino acids, and or about 3 or 4 amino acids in this segment.

As used herein, the terms “amino-rich peptide segment”, “basic peptide segment,” and “positively-charged peptide segment” refer to a peptide sequence of a peptide amphiphile that comprises one or more amino acid residues that have side chains displaying positively-charged acid side chains (e.g., Arg (R), Lys (K), His (H), or non-natural amino acids, or peptidomimetics). A basic peptide segment may optionally contain one or more additional (e.g., non-basic) amino acid residues. Non-natural amino acid residues with basic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides (e.g., a TF-targeting sequence, etc.) exhibits the functionality of the bioactive peptide.

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

### DETAILED DESCRIPTION

Provided herein are compositions comprising a composite of peptide amphiphile (PA) nanofibers and hyaluronic acid (HA) particles, and methods of preparation and use thereof, such as for repair of a cartilage or osteochondral defects.

In some embodiments, provided herein are materials comprising a composite of PA nanofibers and modified HA particles. In some embodiments, when mixed, the two materials form a composite structure, forming an injectable paste that can be further stiffened with the addition of calcium. The resulting material retains its gel properties at high shear strains (100%) and is significantly stiffer than HA alone. On the cellular scale, the material comprises large bundles of HA particles and PA fibers which support cellular growth. In some embodiments, PA fibers bind factors that support tissue repair. In some embodiments, modified HA enhances cartilage growth. In some embodiments, materials herein comprise unique mechanical properties that allow the material to be syringe injected into a cartilage defect or osteochondral defect in arthroscopic surgery and to be retained long enough to support targeted tissue healing.

Compositions herein comprise two primary components; (1) hyaluronic acid particles, and (2) peptide amphiphile nanofiber solutions (e.g., with or without growth factors). In some embodiments, growth factors (GF) and/or GF-binding peptides are displayed on the peptide amphiphile nanofibers and/or embedded with the compositions or peptide amphiphile nanofiber solutions.

In some embodiments, provided herein are hybrid materials comprising both self-assembling and covalent polymer components that comprise injectable scaffolds with suitable physical and bioactive properties for bioregenerative applications. In particular embodiments, provided herein are composite materials combining peptide amphiphile (PA) filaments designed to bind the chondrogenic cytokine transforming growth factor β-1 (TGFβ-1) and crosslinked hyaluronic acid microgels. In some embodiments, the composite produces a mechanically resilient scaffold able to induce hyaline cartilage repair in large-animal joints. Experiments conducted during development of embodiments herein demonstrates that combining the supramolecular and covalent components produced a porous network of bundled PA fibers, which supported chondrogenic differentiation of encapsulated stem cells in response to sustained delivery of TGFβ-1. Four weeks following implantation in shallow osteochondral defects in sheep stifle joints, the scaffold improved cartilage macroscopic scoring of defect fill and integration in both the medial femoral condyle and the trochlear groove. At later time points, histological staining revealed that the hybrid scaffold supported regeneration of hyaline-like tissue. These results demonstrate the potential of supramolecular-covalent hybrid scaffolds to direct cartilage repair in a clinically relevant large-animal model.

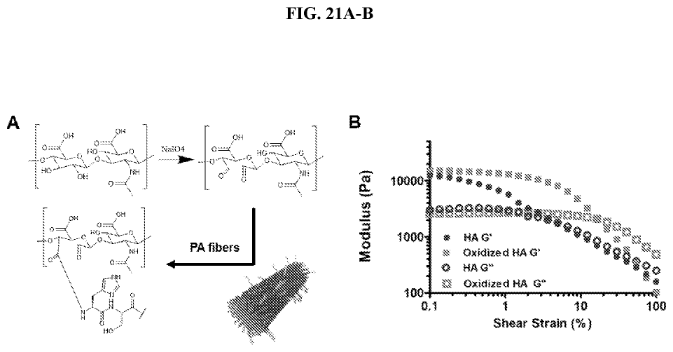

Exemplary compositions described herein use HA particles. In some embodiments, HA particles comprise a modified HA. In some embodiments, HA particles comprise crosslinked HA. In some embodiments, HA is mixed with a cross-linker (e.g. L-lysine methyl ester, etc.) (1-Ethyl-3-(3-dimethylaminopropyl) carbodiimide (EDC), etc.) to produce cross-linked HA. and/or a coupling agent. Some embodiments herein are not limited by the crosslinking chemistry used to crosslink HA. Non-limiting examples of the HA cross-linking agents include glutaraldehyde (GTA), carbodiimides, bisdiazobenzidine, N-maleimidobenzoyl-N-hydroxysuccinimide ester, butanediol diglycidyl ether (BDDE), divinylsulfone, bis(sulfosuccinimidyl) suberate, 3,3″-dithiobis(sulfosuccinimidyl) propionate (DTSSP), and 2-methylsuberimidate (DMS). In some embodiments, HA is provided as a 0.1 wt % to 10 wt % HA solution (e.g., 0.1 wt %, 0.2 wt %, 0.5 wt %, 1.0 wt %, 2.0 wt %, 3.0 wt %, 4.0 wt. %, 5.0 wt %, 6.0 wt %, 7.0 wt %, 8.0 wt %, 9.0 wt. %, 10.0 wt %, or ranges therebetween). In some embodiments, the ratio of lysine cross-linker to coupling agent is varied to control the extent of modification. For example, in experiments conducted during development of embodiments herein, slurries have been produced with using varying concentrations of cross-linker (e.g., 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml, 2.1 mg/ml, 2.2 mg/ml, 2.3 mg/ml, 2.4 mg/ml, 2.5 mg/ml, or more, or ranges therebetween) and coupling agent. (e.g., 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml, 2.1 mg/ml, 2.2 mg/ml, 2.3 mg/ml, 2.4 mg/ml, 2.5 mg/ml, 3.0 mg/ml, 3.5 mg/ml, 4.0 mg/ml, 4.5 mg/ml, 5.0 mg/ml, 6.0 mg/ml, or more, or ranges therebetween). Equal molar concentrations of cross-linker and coupling agent produces modified HA particles bearing both cross-links (e.g., where both amines of the lysine react) and free amines (e.g., where one lysine is coupled to the HA backbone and the other is unmodified). In some embodiments, HA particles are modified by oxidation of all or a portion of the HA within the particles. In some embodiments, FIG. 21 demonstrates exemplary chemistry for (partial) oxidation of HA. In some embodiments, NAIO4 is used as an oxidizing agent to produce (partially) oxidized HA. In some embodiments, oxidation of HA produces an opened D-glucuronic acid displaying a pair of aldehyde moieties. In some embodiments, the aldehydes on the HA are available for crosslinking. In some embodiments, the aldehyde groups are capable of crosslinking to amine groups (e.g., on PA nanofibers, on protein, on cartilage, on bone, etc.). In some embodiments, provided herein are compositions comprising (partially) oxidized HA particles crosslinked to PA nanofibers. In some embodiments, after modification, the cross-linked HA solution is dried, forming a thin film. In some embodiments, dried HA films are reconstituted and rinsed against several changes of water (e.g., for 2 hours, 4 hours, 8 hours, 12 hours, 18 hours, 24 hours, 36 hours, 48 hours, or more, or ranges therebetween) to remove remaining cross-linker and/or coupling agent. In some embodiments, the resulting material is shredded to fine particles (e.g., microparticles, nanoparticles) and dried (e.g., lyophilized).

Peptide amphiphile (PA) nanofiber solutions may comprise any suitable combination of PAs. In some embodiments, at least 0.05 mg/mL (e.g., 0.10 mg/ml, 0.15 mg/ml, 0.20 mg/ml, 0.25 mg/ml, 0.30 mg/ml, 0.35 mg/ml, 0.40 mg/ml, 0.45 mg/ml, 0.50 mg/ml, 0.60 mg/ml, 0.70 mg/ml, 0.80 mg/ml, 0.90 mg/ml, 1.0 mg/ml, or more, or ranges therebetween), of the solution is a filler PA (e.g., without a peptide epitope or other nanofiber surface displayed moiety). In some embodiments, at least 0.25 mg/mL of the solution is a filler PA. In some embodiments, a filler PA is a non-bioactive PA molecule having highly charged glutamic acid residues on the terminal end of the molecule (e.g., surface-displayed end). These negatively charged PAs allow for the gelation to take place between nanofibers via ionic crosslinks. The filler PAs provide the ability to incorporate other bio-active PAs molecules into the nanofiber matrix while still ensuring the ability of the nanofibers solution to gel. In some embodiments, the solutions are annealed for increased viscosity and stronger gel mechanics. These filler PAs have sequences are described in, for example, U.S. Pat. No. 8,772,228 (e.g., C₁₆V₃A₃E₃), which is herein incorporated by reference in its entirety.

In an exemplary embodiment, the HA particles and peptide amphiphile nanofiber solution are mixed at 5 wt % HA particles and 1 wt % PA in neutral pH water. Other ranges (e.g., 1 wt %-20 wt % (e.g., 1, 2, 3, 4, 5, 6, 7, 8, 9 10, 11, 12, 13, 14, 15, 16, 17, 18, 19, 20, or any ranges there between) HA particles; 0.1 wt %-10 wt % (e.g., 0.1, 0.2, 0.3, 0.4, 0.5, 0.6, 0.7, 0.8, 0.9, 1, 2, 3, 4, 5, 6, 7, 8, 9 10, or any ranges there between) PAs) may be employed. In particular embodiments, HA is used from 0 wt % to 20 wt %, and the PAs are used at 0.05 wt % to 3 wt %. In some embodiments, growth factors, such as BMP-2, are mixed into the PA solution before combining with the HA particles but after annealing the PA solution.

In some embodiments, materials herein compromise other biocompatible particles (e.g., in addition to HA particles). Suitable biocompatible polymers for use in the materials herein are selected from the group consisting of: PLA, PLLA, PGA, PGLA, PCL, chitosan, polylactides, polyglycolides, epsilon-caprolactone, polyhydroxyvaleric acid, polyhydroxybutyric acid, other polyhydroxy acids, polytrimethylene carbonate, polyamines, vinyl polymers, polyacrylic acids and their derivatives containing ester, polyethylene glycols, polydioxanones, polycarbonates, polyacetals, polyorthoesters, polyamino acids, polyphosphoesters, polyesteramides, polyfumerates, polyanhydrides, polycyanoacrylates, polyoxamers, polyurethanes, polyphosphazenes, aliphatic polyesters, poly(amino acid), copoly(ether-ester), polyakylene oxalate, polyamides, poly(iminocarbonate), polyoxaester, polyamidoesters, amine group-containing polyoxaester, polyacetal, polyalkanoate, gelatin, collagen, elastine, polysaccharide, alginate, chitin, and combinations thereof.

In some embodiments, particles are of any suitable size and shape. In some embodiments, particles are microparticles and have mean diameters of between 1 μm and 1 mm (e.g., 1 μm, 2 μm, 5 μm, 10 μm, 20 μm, 30 μm, 40 μm, 50 μm, 60 μm, 70 μm, 80 μm, 90 μm, 1 mm, or ranges therebetween). In some embodiments, particles are nanoparticles and have mean diameters of between 1 nm and 1 μm (e.g., 1 nm, 2 nm, 5 nm, 10 nm, 20 nm, 30 nm, 40 nm, 50 nm, 60 nm, 70 nm, 80 nm, 90 nm, 1 nm, or ranges therebetween). In some embodiments, particles are generated using any suitable techniques, such as, freezing (e.g., under liquid N₂), drying, freeze drying, lyophilizing, grinding, milling, exposure to solvent (e.g., ethanol), sieving, and combinations thereof.

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the lipophilic segment (although in some embodiments, alignment of nanofibers is performed via techniques not previously disclosed or used in the art (e.g., extrusion through a mesh screen). Synthesis typically starts from the C-terminus, to which amino acids are sequentially added using either a Rink amide resin (resulting in an —NH₂ group at the C-terminus of the peptide after cleavage from the resin), or a Wang resin (resulting in an —OH group at the C-terminus). Accordingly, embodiments described herein encompasses peptide amphiphiles having a C-terminal moiety that may be selected from the group consisting of —H, —OH, —COOH, —CONH2, and —NH2.

In some embodiments, peptide amphiphiles comprise a hydrophobic (non-peptide) segment linked to a peptide. In some embodiments, the peptide comprises a structural segment (e.g., hydrogen-bond-forming segment, beta-sheet-forming segment, etc.), and a charged segment (e.g., acidic segment, basic segment, zwitterionic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more bioactive groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (e.g., nanofibers)) that bury the lipophilic segment in their core and display the bioactive peptide on the surface. The structural peptide undergoes intermolecular hydrogen bonding to form beta sheets that orient parallel to the long axis of the micelle.

In some embodiments, compositions described herein comprise PA building blocks that in turn comprise a hydrophobic segment and a peptide segment. In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between.) is covalently coupled to peptide segment (e.g., a peptide comprising a segment having a preference for beta-strand conformations or other supramolecular interactions) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segment may comprise natural amino acids, modified amino acids, unnatural amino acids, amino acid analogs, peptidomimetics, or combinations thereof. In some embodiments, peptide segment comprise at least 50% sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises EE.

In some embodiments, peptide amphiphiles comprise a basic peptide segment. For example, in some embodiments, the acidic peptide comprises one or more (e.g., 1, 2, 3, 4, 5, 6, 7, or more) basic residues (R, H, and/or K) in sequence. In some embodiments, the basic peptide segment comprises up to 7 residues in length and comprises at least 50% basic residues. In some embodiments, an acidic peptide segment comprises (Xb)₁₋₇, wherein each Xb is independently R, H, and/or K.

In some embodiments, peptide amphiphiles comprises a structural and/or beta-sheet-forming segment. In some embodiments, the structural segment is rich in H, I, L, F, V, and A residues. In some embodiments, the structural and/or beta-sheet-forming segment comprises an alanine- and valine-rich peptide segment (e.g., AAVV, AAAVVV, or other combinations of V and A residues, etc.). In some embodiments, the structural and/or beta sheet peptide comprises 4 or more consecutive A and/or V residues, or conservative or semi-conservative substitutions thereto. In some embodiments, the structural and/or beta-sheet forming peptide segment comprises 4 or more consecutive non-polar aliphatic residues (e.g., alanine (A), valine (V), leucine (L), isoleucine (I), methionine (M)). In some embodiments, the structural and/or beta-sheet forming peptide segment comprises 2-16 amino acids in length and comprises 4 or more (e.g., 2, 3, 4, 5, 6, 7, 8, 9, 10, 11, 12, 13, 14, 15, 16, or ranges there between) non-polar aliphatic residues.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group. In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH₂(O(CH₂)₂)₂NH, CH2(O(CH₂)₂)₂NHCO(CH₂)₂CCH, etc.). In some embodiments, a spacer or linker further comprises additional bioactive groups, substituents, branches, etc.

Suitable peptide amphiphiles for use in the materials herein, as well as methods of preparation of PAs and related materials, amino acid sequences for use in PAs, and materials that find use with PAs, are described in the following patents: U.S. Pat. Nos. 9,044,514; 9,040,626; 9,011,914; 8,772,228; 8,748,569 8,580,923; 8,546,338; 8,512,693; 8,450,271; 8,236,800; 8,138,140; 8,124,583; 8,114,835; 8,114,834; 8,080,262; 8,076,295; 8,063,014; 7,851,445; 7,838,491; 7,745,708; 7,683,025; 7,554,021; 7,544,661; 7,534,761; 7,491,690; 7,452,679; 7,371,719; 7,030,167; all of which are herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural segment (e.g., comprising VVAA); and (c) a charged segment (e.g., comprising KK, EE, etc.). In some embodiments, any PAs within the scope described herein, comprising the components described herein, or within the skill of one in the field, may find use herein.

In some embodiments, peptide amphiphiles comprise a bioactive moiety. In particular embodiments, a bioactive moiety is the C-terminal or N-terminal most segment of the PA. In some embodiments, the bioactive moiety is attached to the end of the charged segment. In some embodiments, the bioactive moiety is exposed on the surface of an assembled PA structure (e.g., nanofiber). A bioactive moiety is typically a peptide (e.g., growth factor, etc.), but is not limited thereto. In some embodiments, a bioactive moiety is a peptide sequence that binds a peptide or polypeptide of interests, for example, a growth factor. Bioactive peptides and other moieties for achieving functionality will be understood. In some embodiments, bioactive moieties are provided having binding affinity for a target protein (e.g., growth factor). The binding affinity (K_{d}) may be chosen from one of: less than 10 μM, less than 1 μM, less than 100 nM, less than 10 nM, less than 1 nM, less than 100 μM. In some embodiments, bioactive moieties are provided having at least 70% (e.g., 70%, 75%, 80%, 85%, 90%, 95%, 98%, 99%, or ranges therebetween) sequence identity with all or an active portion of a growth factor.

In some embodiments, a peptide amphiphile comprises: (a) a hydrophobic tail comprising an alkyl chain of 8-24 carbons; (b) a structural segment (e.g., comprising VVAA, AAVV, VA, AV, etc.); (c) a charged segment (e.g., comprising KK, EE, EK, KE, etc.), and a bioactive peptide (e.g., growth factor or GF-targeting peptide). In some embodiments, a PA further comprises an attachment segment or residue (e.g., K) for attachment of the hydrophobic tail to the peptide portion of the PA. In some embodiments, the hydrophobic tail is attached to a lysine side chain.

In some embodiments, a peptide amphiphile comprises (e.g., from C-terminus to N-terminus or from N-terminus to C-terminus): bioactive peptide (e.g., growth factor or GF-targeting peptide)-charged segment (e.g., comprising KK, EE, EK, KE, etc.)-structural segment (e.g., comprising VVAA, AAVV, VA, AV, etc.)-hydrophobic tail (e.g., comprising an alkyl chain of 8-24 carbons).

In some embodiments, a peptide amphiphile comprises (e.g., from C-terminus to N-terminus or from N-terminus to C-terminus): bioactive peptide (e.g., growth factor or GF-targeting peptide)-charged segment (e.g., comprising KK, EE, EK, KE, etc.)-structural segment (e.g., comprising VVAA, AAVV, VA, AV, etc.)-attachment segment or peptide (e.g., K)-hydrophobic tail (e.g., comprising an alkyl chain of 8-24 carbons).

In some embodiments, a peptide amphiphile comprises (e.g., from C-terminus to N-terminus or from N-terminus to C-terminus): bioactive peptide (e.g., growth factor or GF-targeting peptide)-KKAAVV (K)-hydrophobic tail (e.g., comprising an alkyl chain of 8-24 carbons). In some embodiments, the hydrophobic tail is attached to the (K) sidechain.

In some embodiments, provided herein are nanofibers and nanostructures assembled from the peptide amphiphiles described herein. In some embodiments, a nanofiber is prepared by the self-assembly of the PAs described herein. In some embodiments, a nanofiber comprises or consists of PAs displaying a growth factor or GF-targeting peptide. In some embodiments, the growth factor or GF-targeting peptide peptides are displayed on the surface of the nanofiber. In some embodiments, in addition to PAs displaying GF-targeting peptides, filler PAs are included in the nanofibers. In some embodiments, filler PAs are peptide amphiphiles, as described herein (e.g., structural segment, charged segment, hydrophobic segment, etc.), but lacking a bioactive moiety. In some embodiments, the filler PAs and growth factor PAs or GF-targeting PAs self-assemble into a nanofiber comprising both types of PAs. In some embodiments, nanostructures (e.g., nanofibers) assembled from the peptide amphiphiles described herein are provided.

In some embodiments, PA nanofibers and/or solutions comprising PA nanofibers provide a scaffold and/or environment for supporting growth factors (or other bioactive agents), but are not covalently linked to such growth factors (or other bioactive agents).

In some embodiments, nanostructures are assembled from (1) PAs bearing a bioactive moiety (e.g., growth factor or GF-targeting peptide) and (2) filler PAs (e.g., PAs not-labeled or not displaying a bioactive moiety, etc.). In some embodiments, nanostructures (e.g., nanofibers) comprise: (i) less than 50% (e.g., 49%, 45%, 40%, 35%, 30%, 25%, 20%, 15%, 10%, 5%, 4%, 3%, 2%, 1%, or any ranges there between) PAs bearing a bioactive moiety (e.g., growth factor or GF-targeting peptide moiety). In some embodiments, nanostructures (e.g., nanofibers) comprise and at least 2% (e.g., 2%, 3%, 4%, 5%, 10%, 15%, 20%, 25%, 30%, 35%, 40%, 45%, 50%, 55%, 60%, 65%, 70%, 75%, 80%, 85%, 90%, 95%, 98%, or any ranges there between) PAs bearing a bioactive moiety (e.g., growth factor or GF-targeting peptide moiety). In some embodiments, nanofibers comprise at least 50% (e.g., 50%, 55%, 60%, 65%, 70%, 75%, 80%, 85%, 90%, 95%, 98%, or any ranges there between) filler peptide amphiphiles. In some embodiments, the ratio of PAs bearing a bioactive moiety to filler PAs determines the density of bioactive moieties (e.g., growth factor or GF-targeting moiety) displayed on the nanostructure surface.

In some embodiments, the materials described herein find use in the delivery of growth factors or other bioactive agents for the repair of tissue defects and/or regeneration of tissue (e.g., cartilage). Suitable agents for use in embodiments herein include platelet-derived growth factor (PDGF), bone morphogenic proteins (e.g., BMP-1, BMP-2, BMP-4, BMP-6, and BMP-7); members of the transforming growth factor beta (TGF-β) superfamily including, but not limited to, TGF-β1, TGF-β2, and TGF-β3; growth differentiation factors (GDF1, GDF2, GDF3, GDF5, GDF6, GDF7, myostatin/GDF8, GDF9, GDF10, GDF11, and GDF15); vascular endothelial growth factor (VEGF); fibroblast growth factor (FGF); etc. These agent, or others, may be covalently linked to PAs, non-covalently associated with peptides displayed on PA nanofibers, embedded within a PA nanofiber matrix, embedded within the composite compositions described herein, etc.

Embodiments herein find use in facilitating cartilage regeneration and repair of cartilage defects and/or osteochondral defects. Cartilage is affected by various diseases including congenital morphological anomalies such as cleft lip and palate, trauma to joint, large deficits after surgery for tumors or cancers, ageing-related diseases such as osteoarthritis, and inflammatory diseases such as rheumatoid arthritis. Once the cartilage tissues are damaged due to those disorders, it becomes difficult to maintain the morphology of face or body, as well as resulting in deterioration of daily activities. Cartilage tissues have limited capacity for self-repair. Cartilage diseases have traditionally been treated with transplantation of the autologous cartilage, or replacement with artificial joints; there are issues with those types of treatment, such as durability, infection, and invasiveness of donor sites. For cartilage regeneration, the compositions localize and maintain cells and growth factors within the defect site. In some embodiments, the compositions herein are implanted through minimally invasive means and biodegrade into amino acids and lipids that are safely cleared by the body.

### Experimental

### Example 1

HA (molecular weight ˜1−2*10⁶) was modified by mixing a 1 wt % solution of HA with 1-lysine methyl ester as a cross-linker and 1-Ethyl-3-(3-dimethylaminopropyl) carbodiimide (EDC) as a coupling agent. The ratio of lysine cross-linker to EDC is varied to control the extent of modification. Slurries were produced using varying concentrations of cross-linker and EDC (Table 1), but experiments conducted during development of embodiments herein primarily focused on equal molar concentrations of each; this produces modified HA particles bearing both cross-links where both amines of the lysine react and free amines where one lysine is coupled to the HA backbone and the other is unmodified. After mixing, the polymer solution is dried, forming a thin film; this concentrates the solution during the cross-linking reaction. After drying, the films are reconstituted and rinsed against several changes of water for at least 24 hours to remove all remaining cross-linker and EDC. The hydrogel is then shredded to produce fine particles and lyophilized.

TABLE 1
Combinations of EDC and L-lysine methyl ester tested (mg/ml)

| EDC (mg/ml) | L-lysine methyl ester |
|---|---|
| 0.3 | 0.175 |
| 0.45 | 0.39375 |
| 0.6 | 0.7 |
| 0.9 | 1.575 |
| 0.312 | 4.732 |
| 0.6 | 0.7 |
| 0.9 | 0.7875 |
| 1.5 | 1.09375 |
| 0.3 | 0.35 |
| 1.2 | 1.4 |

PAs were synthesized using solid phase peptide synthesis followed by HPLC purification. Experiments conducted during development of embodiments herein primarily utilized the PA sequence palmitoyl-VVVAAAEEE; however, other self-assembling sequences can be used, including PA fibers with positive charge such as palmitoyl-VVVAAAKKK. In some embodiments, the slurry material is made with any variation of Nanofiber comprising: aliphatic tail (e.g., C16)-structural peptide segment (e.g., VVVAAA, VVAA, etc.)—charged peptide segment (e.g., EEE, KKK, etc.)-optional bioactive sequence (e.g., IKVAV, RGDS, etc.)

To produce a slurry, the lyophilized HA particles are reconstituted with a PA solution. Various concentrations and ratios of HA to PA were tested (e.g., 0.1% HA, 0.2% HA, 0.5% HA, 0.75% HA, 1.0% HA, 1.5% HA, 2.0% HA, 2.5% HA, 3.0% HA, 3.5% HA, 4.0% HA, 4.5% HA, 5.0% HA, 6.0% HA, 7.0% HA, 8.0% HA, 9.0% HA, 10% HA, 12% HA, 15% HA, or more or ranges therebetween). Increased PA makes the material stiffer, but more susceptible to failure at high shear. Increasing modified HA makes the material more strain resistant but increases swelling which can force the material out of the defect as it expands. For animal studies, a solution of 2% PA and 4% modified HA was used. When calcium is added, the formulation used has a stiffness of about 2 kPa at low strain and maintains a stiffness of at least 1 kPa above 5% strain is gel-like as to 100% strain.

For surgical application, the slurry is prepared as described above. If loaded with growth factor, the protein was mixed well in the PA solution prior to addition to HA. The paste was then packed into a syringe and ejected through an 18-gauge needle into the defect. Following application, a 50 mM calcium chloride solution is dripped over the implanted material through a syringe to induce stiffening. For surgical studies, 3 mm deep 7 mm diameter critical-sized defects were made in the trochlea of the need or the femoral condyle of a sheep model. The 3 mm depth was chosen because it is similar to the thickness of human knee cartilage. After one week, fluorescent imaging and histological staining confirmed strong retention of the PA when delivered in this form. The defects remained filled with material and cells had infiltrated the scaffold.

### Example 2

### Materials and Methods

PA HA hybrid preparation and characterization: PA molecules were synthesized using an automated synthesizer and purified by reverse-phase high-performance liquid chromatography. Prior to use, we dissolved each PA component, mixed the solutions volumetrically, bath sonicated, and then heat treated the mixture. To produce HA particles, HA powder was dissolved in water, crosslinked the polymer with l-lysine methyl ester, dried the solution to form a film, rehydrated and shredded the film to form gel particles, and then lyophilized the particles. The extent of crosslinking was determined by measuring the amount of crosslinker retained using elemental analysis with incomplete crosslinks measured using a ninhydrin assay. Viscoelastic properties were measured by shear rheometery and measured swelling by comparing wet weight and dry weight of gels at several times after addition to buffer. Degradation of the hybrid material in a hyaluronidase solution was assessed using a carbazole assay to measure uronic groups in the supernatant (Ref. 57; incorporated by reference in its entirety). Self-assembly of the material was evaluated using SEM and SAXS/MAXS/WAXS, and cell encapsulation was evaluated by SEM and by immunocytochemistry staining of PA, nuclei, and actin. TGFβ-1 release by the gels into a buffer supernatant was measured using an enzyme-linked immunosorbent assay. For differentiation experiments, gels were loaded with hMSCs; cultured the gels in chondrogenic media without TGFβ-1, with TGFβ-1 added to the media, or with TGFβ-1 added to the gels prior to encapsulation for 4 weeks; and sGAG content was determined using a dimethyl methylene blue assay (Ref. 58; incorporated by reference in its entirety).

Osteochondral defect filling and regeneration: Skeletally mature female sheep were anesthetized and arthrotomy was performed bilaterally. One 3-4 mm deep and 7 mm diameter osteochondral defect was made in the medial femoral condyle of each stifle and one defect of the same dimensions in the femoral trochlear groove of each stifle. In one stifle, both defects were immediately filled with the PA/HA hybrid with 1 μg/mL TGFβ-1 preloaded as indicated. In the contralateral stifle, saline was immediately added to both defects, which was supplemented with TGFβ-1 at the same concentration as used in the material treated stifle. A calcium chloride solution was then dripped on top of each defect. The arthrotomies and allowed the sheep to stand on the hindlimbs. Samples of each sheep's synovial fluid was antiseptically drawn immediately prior to surgery and one week following surgery. After euthanasia, gross appearance of the defect areas was assessed the ICRS macroscopic scoring system (Ref. 59; incorporated by reference in its entirety) (Table 4). The samples were then fixed in formalin, decalcified the samples and prepared sections which were stained with H&E, with safranin O, or immunohistochemically for collagen II. Sections were scored using a modified O'Driscoll scoring system (Ref. 60; incorporated by reference in its entirety) (Table 5).

TABLE 4
ICRS macroscopic scoring system criteria (59).

| | |
|---|---|
| Cartilage repair assessment ICRS | Score |

Degree of defect repair

| | |
|---|---|
| In level with surrounding cartilage | 4 |
| 75% repair of defect depth | 3 |
| 50% repair of defect depth | 2 |
| 25% repair of defect depth | 1 |
| 0% repair of defect depth | 0 |

Integration to border zone

| | |
|---|---|
| Complete integration with surrounding cartilage | 4 |
| Demarcating border <1 mm | 3 |
| ¾th of graft integrated, ¼th with a notable border >1 mm width | 2 |
| ½ of graft integrated with surrounding cartilage, ½ with a notable border >1 mm | 1 |
| From no contact to ¼th of graft integrated with surrounding cartilage | 0 |

Macroscopic appearance

| | |
|---|---|
| Intact smooth surface | 4 |
| Fibrillated surface | 3 |
| Small, scattered fissures or cracks | 2 |
| Several, small or few but large fissures | 1 |
| Total degeneration of grafted area | 0 |

TABLE 5
O'driscoll histology scoring criteria (60).

| | |
|---|---|
| Characteristics | Score |

Nature of predominant tissue
Cellular morphology

| | |
|---|---|
| Hyaline cartilage | 4 |
| Incomplete differentiated mesenchyme | 2 |
| Fibrous tissue or bone | 0 |

Safranin O staining of matrix

| | |
|---|---|
| Normal | 3 |
| Moderate | 2 |
| Slight | 1 |
| None | 0 |

Structural characteristics
Surface regularity

| | |
|---|---|
| Smooth and intact | 3 |
| Superficial horizontal lamination | 2 |
| Fissures 25%-100% of thickness | 1 |
| Severe disruption, fibrillation | 0 |

Structural integrity

| | |
|---|---|
| Normal | 2 |
| Slight disruption, cysts | 1 |
| Severe disintegration | 0 |

Thickness

| | |
|---|---|
| 100% of normal adjacent cartilage | 2 |
| 50%-100% of normal cartilage | 1 |
| 0%-50% of normal cartilage | 0 |

Freedom from cellular changes of degeneration
Hypocellularity

| | |
|---|---|
| Normal cellularity | 3 |
| Slight hypocellularity | 2 |
| Moderate hypocellularity | 1 |
| Severe hypocellularity | 0 |

Chondrocyte clustering

| | |
|---|---|
| No clusters | 2 |
| <25% of cells | 1 |
| 25%-100% of cells | 0 |

Peptide amphiphile synthesis: Peptides synthesis was performed at the Peptide Synthesis Core at the Simpson Querrey Institute via standard Fmoc solid-phase peptide synthesis chemistry on a Rink MBHA resin using a CEM Liberty microwave-assisted synthesizer. For flourophore-labelled PA, a methtrityl protecting group was first removed from the lysine using 1:5:94 solution of trifluoroacetic acid (TFA): triisopropylsilane (TIS): dichloromethane (DCM). After washing with DCM, the carboxytetramethyrhodamine (TAMRA) was coupled using 1.2 equivalents of TAMRA, 1.2 equivalents of PyBOP (benzotriazol-1-yl-oxytripyrrolidinophosphonium hexafluorophosphate), and 8 equivalents of N,N-diisopropylethylamine overnight. Peptides were cleaved from the resin using a 95:2.5:2.5 solution of TFA: TIS: DCM for three hours followed by concentration and precipitation dropwise into cold diethyl ether. The precipitate was resuspended in 0.1% ammonium hydroxide in water and passed through a 0.2 μm syringe filter prior to purification. Peptides were purified by standard reverse phase high-performance liquid chromatography (HPLC) on a Waters Prep150 instrument (Milford, MA, USA) against a water/acetonitrile gradient with 0.1% ammonium hydroxide. Eluted fractions were collected and assessed using Agilent 6520 QTOF liquid chromatograph-mass spectrometer (LC-MS; Santa Clara, CA, USA). Confirmed fractions were pooled, acetonitrile was removed by rotary evaporation, and solutions were frozen and dried via lyophilization Purity of the product was confirmed by LCMS using a Phenomenex Gemini C18 column over a 5% to 95% water to acetonitrile gradient with 0.1% ammonium hydroxide.

HA particle synthesis: HA sodium salt from Streptococcus equi with a 1.5-1.8 MDa molecular weight (Sigma-Aldrich) was obtained and autoclaved for sterilization. In a biological safety cabinet, sterile water was added to the powder to 1.25 wt % and the solution was agitated overnight to dissolve the material. A solution was prepared in sterile water of 3 mg/ml N-(3-Dimethylaminopropyl)-N′-ethylcarbodiimide hydrochloride (EDC; Sigma-Aldrich) and 3.5 mg/mL l-lysine methyl ester dihydrochloride (Sigma-Aldrich), sterile filtered, and added to the HA solution to 20% of the total volume. The mixed solution was agitated for 30 min and then poured into cell culture flasks to cover their surface at 1 ml/cm². The solution was allowed to dry in the biological safety cabinet over 72 h. The resulting film was rehydrated with sterile water and washed by in an excess of water under agitation for at least 24 h with 3 changes of water to remove excess crosslinker. The gel was transferred to a Magic Bullet blender and blended for a total of 5 min. The resulting suspension was frozen and lyophilized.

Ninhydrin assay: 1 mg HA was dissolved in 1 mL of water for 1 h to which was added 500 μL ninhydrin reagent (Sigma-Aldrich). Samples were placed in boiling water for 10 min and transferred to a microplate. Absorbance at 570 nm was read using a Cytation 3 microplate reader. Absorbance was compared to a standard curve of l-lysine methyl ester dissolved at known concentrations to calculate primary amine content.

Gel preparation: The diluent PA molecule and TGFβ-1 binding PA molecule were each suspended in sterile water to 19 mM for materials characterization or in vivo experiments and to 38 mM for in vitro biological experiments. The pH of the solution was adjusted to 7 using a sterile filtered solution of 1 M sodium hydroxide. For experiments where a fluorescently tagged PA was used, the fluorescently tagged PA was dissolved to its stated final concentration and then the diluent PA or binding PA powders were resuspended in the fluorescent PA solution. Solutions of the diluent PA and binding PA were mixed at a 9:1 ratio, sealed in a sterile tube, and bath sonicated for 1 h. The solutions were heated in a water bath to 80° C. for 1 h and allowed to cool in the bath overnight. Immediately prior to further use, PA solutions were diluted by 10% with either sterile water or a solution of recombinant human TGFβ-1 to achieve the stated protein concentration and a molar PA concentration equivalent to 2 wt % diluent PA. To produce the composite gel, the PA solution was added to achieve the reported concentration (4 wt % for all biological experiments). The resulting slurry was mixed with a spatula and spun down with a microcentrifuge several times to initiate hydration. Samples were allowed to hydrate on ice for 1 h prior to use.

Transmission electron microscopy: A heat-treated PA solutions was diluted 10-fold in water and 10 μL of the resulting solution was placed on copper mesh TEM grid (Electron Microscopy Science) for 3 min, rinsed twice with water, stained twice with 2 wt % uranyl acetate, rinsed twice more with water, and dried for 10 min. The grid was imaged using a FEI Spirit G2 transmission electron microscope.

Rheology: Measurements were performed on an Anton Paar MCR302 rheometer with 25 mm parallel-plate fixture. 90 μL of the PA solution or the PA/HA hybrid slurry was placed on the measurement stage and 30 μL of a 50 mM CaCl₂)/75 mM NaCl gelling solution was placed on the upper fixture. The fixture was lowered to a gap of 0.5 mm for 5 min and then lowered further to 0.25 mm for another 5 min during which time a 0.1% oscillatory strain was applied with a 10 rad/s angular frequency. A strain sweep was then performed at the same angular frequency from 0.1% to 100% strain.

HA degradation assay: HA only samples were prepared by dissolving the HA powder as received or following crosslinking to 4 wt % in 100 μL sterile water. PA/HA hybrid samples were prepared to 100 μL using HA as received or following crosslinking to 4 wt %. 5 replicates were prepared of each sample were prepared in conical microcentrifuge tubes. To each sample, 50 μL of a 25 mM CaCl₂) and 0.1% bovine serum albumin (BSA) in tris-buffered saline (TBS) gelling solution was added for 1 h. Then, 850 μL of a 100 U/mL solution of hyaluronidase Type I-S (Bovine source; Sigma-Aldrich) in 10 mM CaCl₂) and 0.1% BSA supplemented TBS was added to each sample and incubated at 37° C. Every 2 d, the tubes were inverted twice and 200 μL of the supernatant was removed and flash frozen. After 10 d, 500 μL PBS was added to each tube and the remaining material was triturated and agitated for 1 h to break apart the remaining gel. The degradation of HA was quantified by measuring the concentration of uronic acid functional groups in the supernatant or in remaining gel. Samples were thawed and batch sonicated for 1 h. A standard curve was prepared by serially diluting a 1 mg/mL glucuronic acid glucuronic acid γ-lactone in water. 25 μL of sample or standard was placed in a 96 well plate in triplicate and solutions were diluted 1:1 in saturated benzoic acid. To each well was added 100 μL fuming sulfuric acid supplemented with 25 mM sodium tetraborate. Samples were heated to 100° C. in a sand bath for 10 min and then allowed to cool to ambient temperature. To each well was added 25 μL 0.125% carbazole in absolute ethanol and samples were heated again to 100° C. for 10 min and cooled to ambient temperature. Optical absorbance of each well was read at 550 nm using a Biotek Cytation 3 microplate reader and uronic acid concentration was determined based on the glucuronic acid γ-lactone standard curve. Total degradation was normalized to the fraction of uronic acid groups released at each timepoint relative to the sum of the total detected during the degradation timepoints and total remaining in the gel after 10 d.

Swelling assay: 50 μL PA solution or PA/HA hybrid was added to a conical microcentrifuge tube, and 50 μL of a 50 mM CaCl₂)/75 mM NaCl gelling solution was added to each tube. After 1 h, excess gelling solution was removed and 950 μl water was added to each tube. At each timepoint, five gels of each sample were removed from their tube and transferred to a pre-weighed microcentrifuge tube. The weight was recorded and then the gels were frozen with liquid nitrogen, lyophilized, and weighed again to determine the volumetric swelling ratio. The final ratio was compared to the ratio of water to material as prepared to determine the volume increase at each timepoint. Swelling data were plotted as a function of time and fit to a first-order kinetics model.

Scanning electron microscopy: Cell-free samples were prepared by gelling PA solution or the PA/HA hybrid using a 50 mM CaCl₂)/75 mM NaCl gelling solution for 1 h. For cell-encapsulating gels, the media was removed and samples were fixed for 30 minutes with 2% paraformaldehyde/2.5% glutaraldehyde in 150 mM NaCl followed by 3 washes with 150 mM NaCl. Gels were transferred to a stainless-steel cage and dehydrated through a series of water-ethanol solutions to absolute ethanol. The samples were then dried at the critical point of CO₂ using Samdri-795 Critical Point Dryer (Tousimis). Prior to imaging, samples were coated with 25 mm osmium using Filgen OPC-60A plasma coater. Imaging was performed using a Gemini 1525 SEM at an accelerating voltage of 3.0 kV for cell-free samples or a Hitachi SU8030 SEM at an accelerating voltage of 2.0 kV for cell-encapsulating samples.

X-ray scattering: SAXS, MAXS, and WAXS measurements were performed simultaneously using beamline 5ID-D in the Dupont-Northwestern-Dow Collaborative Access Team Synchrotron Research Center at the Advanced Photon Source at Argonne National Lab. To prepare the samples, an adhesive silicone spacer was placed on a piece of Kapton tape and the PA solution or the PA/HA hybrid was placed within the spacer. A second piece of tape was the flattened above the tape to seal the sample between two Kapton windows. The sample was exposed to 17 keV x-rays five times for 1 s and two-dimensional scattering patterns were recorded with using three Rayonix CCD detectors. The patterns were converted to one-dimensional intensity profiles by azimuthal integration using the data reduction program FIT2D and plotted against the wave vector q=(4π)sin(θ/2) where d=2π/q.

Protein release: Solutions of 100% diluent PA or 90% diluent PA and 10% binding PA were heat treated at 17 mM and combined 9:1 with a solution of 10 μg/ml recombinant human TGFβ-1 (eBioscience) and 0.1% BSA and allowed to mix for 1 h on ice. As a control, the TGFβ-1 solution was diluted 10 times in water. 100 of each μl PA was combined with HA to 4 wt % and allowed to hydrate for 1 h on ice in a conical microcentrifuge tube. To each PA/HA hybrid or control sample was added 50 μL of a 25 mM CaCl₂) and 0.1% BSA in TBS gelling solution and samples were incubated at 37° C. for 1 h. After gelling, each tube was filled with 400 μL 10 mM CaCl₂) and 0.1% BSA supplemented TBS and incubated at 37° C. At each timepoint, 200 μl of the buffer solution was removed and replaced and samples were stored at −80° C. After 21 days, samples were removed, 400 μL PBS was added to each tube followed by triteration and agitation for 1 h to remove the remaining growth factor from the gels. The TGFβ-1 concentration of each sample was quantified using a Ready-Set-Go Human/Mouse TGFβ-1 ELISA kit (Invitrogen) according to the manufacturers instruction and normalized to the gel-free control sample. At each timepoint, the protein released was normalized to the sum of the total protein released and protein remaining within each gel.

Cellular encapsulation: MSCs were obtained from Lonza and maintained in MSC growth media (Lonza). Cells were used for experiments prior to passage 6. Cells were trypsinized and resuspended in growth media for counting followed by centrifugation and resuspension to 4 million cells/mL in DMEM. Cells were mixed 1:1 volumetrically with PA solutions heat treated at 38 mM and diluted 10% with water or a 20 μg/mL solution of recombinant human TGFβ-1 (R&D Systems) as indicated and incubated together for 1 h on ice. 50 μL PA/cell solutions were added to a conical centrifuge tube containing a 2 mg HA, mixed with a spatula, and incubated for 1 h at 37° C. To each sample was added 50 μL sterile gelling solution (50 mM CaCl₂)/75 mM NaCl) and excess gelling solution was removed after 30 min incubation at 37° C. Each tube was filled with 770 μL chondrogenic media containing DMEM supplemented with 4.5 g/L glucose, 1% penicillin/streptomycin, 1% insulin/human transferrin/selenous acid premix, 0.1 mM 1-ascorbic acid 2-phosphate sesquimagnesium salt hydrate, and 100 nM dexamethasone. Media was supplemented with freshly thawed aliquots of TGFβ-1 to 10 ng/ml for soluble TGFβ-1 experimental groups. Half the media was removed and replaced three times per week during the culture period.

Cytotoxicity assay: Cells were cultured encapsulated in PA only gels or PA/HA hybrid gels for 24 h in TGFβ-1 free media. Media was removed and replaced with 2 μg/mL calcein and 5 μg/mL propidium iodide in TBS. Samples were incubated at 37° C. for 1 h and then washed 3 times with TBS. Gels were removed and placed on a coverglass immediately prior to imaging. Three regions of each of three gels per experimental group were imaged Nikon W1 Dual Cam Spinning Disk Confocal Microscope and cells were counted using the MATLAB Image Processing Toolbox.

Immunohistochemistry: Gels were fixed with 4% paraformaldehyde in 150 mM NaCl for 20 min and washed 3 times 150 mM NaCl. Gels were then snap frozen in liquid nitrogen and cut into sections with a razor blade. Sections were laid flat on a microscope coverglass and cells were permeabilized for one h with a blocking buffer containing 150 mM NaCl, 2% goat serum and 0.2% Triton X-100. Sections were stained overnight with a solution containing rhodamine-phalloidin at 100× dilution, and Hoechst 33258 at 100× dilution in blocking buffer. Samples were rinsed three times, coverslipped, and imaged using a Nikon A1R laser confocal microscope.

Sulfated glycosaminoglycan quantification assay: Cells were encapsulated in either PA only gels or PA/HA hybrid gels. For each material, 5 sample replicates were prepared and maintained in each of three conditions. These were (1) gels cultured without TGFβ-1 supplementation, (2) gels cultured in TGFβ-1 supplemented media, and (3) gels prepared containing 1 μg/mL TGFβ-1 and cultured without further TGFβ-1 supplementation. After 4 weeks of culture, the total TGFβ-1 added via media supplementation in the soluble TGFβ-1 group equaled the total TGFβ-1 encapsulated in the gels in the growth factor encapsulation group (50 ng). At 4 weeks, the media was removed and to each tube was added 250 μL digestion buffer containing 0.2 mg/mL papain (Sigma-Aldrich), 10 mM l-cysteine, 60 mM NaH₂PO₄, and 40 mM NA₂HPO₄. Gels were broken apart via trituration, transferred to screw-top conical microcentrifuge tubes, and incubated for 18 h at 60° C. A standard curve was prepared by serially diluting a 250 μg/mL solution of CS from bovine trachea in the digestion buffer. After digestion, 25 μL of each sample or standard solution was added in triplicate to a 96 well plate and diluted 1:1 in the digestion buffer. To each well was added 250 μL of a detection solution reported containing mg/ml NaCl, 3 mg/mL glycine, 10 mM acetic acid, and 16 μg/mL dimethyl methylene blue, pH 1 as previously reported (58). Optical absorbance for each well at 525 nm was recorded using a Cytation 3 microplate reader (BioTek). Total DNA concentration for each sample was measured using a Quant-iT PicoGreen assay kit (Invitrogen) according to the manufacturer's protocol and total sGAG content was normalized to DNA content for each sample.

Preparation of material for surgical implant: Heat treated PA solutions prepared at 19 mM were combined with a 10 μg/mL TGFβ-1 solution (R&D Systems) dissolved in a 0.1% BSA, 4 mM HCl buffer or with the buffer alone at a 9:1 ratio. The combination was mixed by pipetting and kept on ice for 1 h. 900 μL of the mixed solution was added to 36 mg of crosslinked HA particles prepared under sterile conditions and allowed to hydrate for 1 h. The hybrid material was packed into a 1 mL syringe with a 19-gauge needle attached and kept on ice until use during surgery.

Animal study design: Animal work was performed at the University of Wisconsin, School of Veterinary Medicine, and all procedures were approved by the Institutional Animal Use and Care Committee. Thirty-six mature female sheep ranging in age from 2-7 (mean=3.7±1.7) years weighing between 60 and 100 (mean=74±8.3) kg were used in this study. Animals were divided into 6 groups: (1) 1 pilot sheep sacrificed at 1 week to assess material retention, (2) 6 sheep sacrificed at 4 weeks treated with PA and TGFβ-1, with 1 sheep sacrificed prior to 4 weeks due to patellar luxation and excluded from the analysis, (3) 6 sheep sacrificed at 4 weeks with PA, but without TGFβ-1 treatment, (4) 14 sheep sacrificed at 12 weeks with PA and TGFβ-1 treatment, with 4 sheep sacrificed prior to 12 weeks due to patellar luxation and excluded from analysis, (5) 6 sheep sacrificed at 12 weeks with PA but without TGFβ-1 treatment, with 2 sacrificed prior to 12 weeks due to patellar luxation and excluded from analysis, and (6) two sheep sacrificed at 24 weeks with PA and TGFβ-1 treatment.

Surgical technique: Prior to surgery the sheep were fasted for 24 h, and withheld water for 12 h. Sheep were administered a dose of Xylazine (0.1-0.22 mg/kg IM) in their pen to sedate them for transport to the surgical suite as well as a pre-operative dose of buprenorphine (0.0006-0.01 mg/kg IM) for analgesia. Sheep were then induced with ketamine-midazolam (2-10 mg/kg-0.1-0.3 mg/kg IM) and immediately intubated. Once intubated the sheep were maintained on isoflurane (0-4%) in 100% O₂. At this time, a dose of Procaine Penicillin-G (10,000-20,000 IU/kg) IM was administered. Using aseptic techniques, a medial stifle arthrotomy was performed on both limbs of each sheep. In each stifle, one osteochondral defect was made in the medial femoral condyle and a second was made in the femoral trochlear groove using a Stryker EHD variable speed surgical drill and a cannulated drill bit. For the 24-week group, defects were made in the condyle only and not the trochlear groove. Defects were 3-4 mm deep and 7 mm in diameter which is previously reported to be critical sized for sheep osteochondral defects. For the one-week pilot group, all defects were filled with the PA/HA material, which included TAMRA-tagged PA filaments. For TGFβ-1 treated groups, each defect on one side was filled with the TGFβ-1 supplemented PA/HA material which was ejected from the syringe and packed into the defect space, while each defect on the control side was filled with 1 μg/mL TGFβ-1 in saline solution. For the no growth factor experimental groups, the PA/HA material without added TGFβ-1 was used to fill the defects on one side in the same manner while defects on the opposite side were filled with saline solution only. After defect filling with PA/HA or saline solution, approximately 30 μL of a sterile 50 mM CaCl₂), 75 mM NaCl gelling solution was added dropwise on top of each defect. Arthrotomies were closed in a routine manner after thoroughly lavaging the joint with physiological saline Immediately after surgery, all sheep were individually housed in a small pen that allowed for limited movement for the first 3 weeks post-surgery. After this the sheep were moved to group housing and allowed to move freely. In the 1-week group, one hindlimb was kept in a sling, but all other animals were allowed to walk freely on both hindlimbs.

Synovial fluid sample collection and analysis: For the 12-week survival group, joint fluid was collected via a joint tap prior to arthrotomy using a 3 mL syringe with an 18-gauge needle under an aseptic condition. Additional joint fluid samples were collected via a sterile joint tap at 1 week, 2 weeks, and 4 weeks post-surgery, and immediately following sacrifice. Samples were stored at −80° C. and thawed prior to evaluation. Cytokine concentration was determined for pre-surgery and 1-week post-surgery samples using a MILLIPLEX Bovine Cytokine/Chemokine 15 plex panel (Millipore-Sigma) using the Luminex xPONENT 3.1 instrument according to the manufacturer's instructions. The results were analyzed using the Milliplex Analyst software. The analytes for interleuken 1α (IL-1α), interleuken 6 (IL-6), interleuken 8 (IL-8), interleuken 10 (IL-10), macrophage inflammatory protein 1α (MIP-1α), interleuken 36 receptor agonist (IL-36-RA), interferon gamma-induced protein 10 (IP-10), monocyte chemoattractant protein 1 (MCP1), tumor necrosis factor α (TNFα), and vascular endothelial growth factor (VEGF) were reliably detected and their concentrations are reported. The analytes interleukin-1ß, interleukin-4, interleukin-17A, interferon-γ, and macrophage inflammatory protein-β were not detected in the samples.

Cartilage sample collection: After euthanasia, operative joints were harvested and the gross appearance was documented with digital photographs. The gross appearance of both condylar and trochlear defect areas was assessed subjectively using the ICRS macroscopic scoring system (Table 4) (Ref. 59; incorporated by reference in its entirety) by experienced evaluators blinded to treatment group. Mean scores between the PA/HA treated and limbs control limbs for the 4-week and 12-week survival groups were compared using a Mann-Whitney test. The operated medial condyles and trochleae in each group then were cut using a band saw into small bone blocks, which included the defect and its associated underlying subchondral bone.

Histological staining and evaluation: The bone blocks were fixed in 10% neutral buffered formalin, and subsequently decalcified using an EDTA/Sucrose decalcifying solution (20% EDTA in 5% sucrose). After decalcification, the blocks were cut in half through the center of the defect using a razor blade and both pieces were embedded in the same block of paraffin. Successive 5 μm thick sections were prepared and stained with H&E to evaluate general morphology of the grafted site and with Safranin-O to evaluate the proteoglycans of the ECM.

All sections were scored by the same 3 investigators who performed the macroscopic scoring. Scorers were blinded to group assignment and used a modified O'Driscoll scoring system (Table 5) (Ref. 60; incorporated by reference in its entirety). Mean scores for treated and control limbs for the 4-week and 12-week survival groups were compared using a Mann-Whitney test. Following scoring, stained sections were imaged using a TissueGnostics microscope. Unstained sections from the pilot study where TAMRA-tagged PA filaments were used were imaged using a Nikon Ti2 Widefield microscope.

Immunohistochemistry staining and evaluation: Immunohistochemical staining for Collagen II was carried out using a rabbit polyclonal antibody (abcam) and a Ventana Discovery Ultra automated staining machine. Deparaffinization was carried out on the instrument, as was heat-induced epitope retrieval in the form of cell conditioning with CC2 buffer (Ventan), a citrate-based buffer, for approximately 8 min at 91° C. (as recommended by the manufacturer). The slides were incubated with the primary antibody, Collagen II, diluted 1:50 in DaVinci Green (BioCare Medical) for 1 h at 37° C. They were next rinsed with Reaction Buffer (Ventana) and then incubated with Omni-Map anti-Rabbit HRP (Ventana) 16 min at 37° C. A second rinse with Reaction Buffer was performed, and Chromo Map DAB detection (Ventana) was applied for a preset time. The slides were removed from the instrument and rinsed with dawn dish soap and warm tap water, then rinsed with dH₂O. Harris hematoxylin counterstain 1:5 was applied for 45 s, and blued with LiCO₂-8 dips. A final rinse with deionized water was performed prior to dehydration with xylene and coverslipped.

### Results

### Preparation and Characterization of PA HA Hybrid Gels

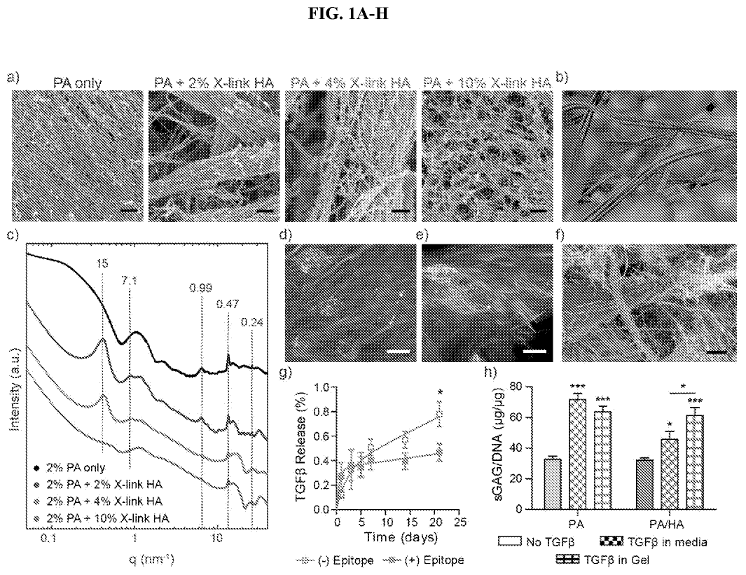

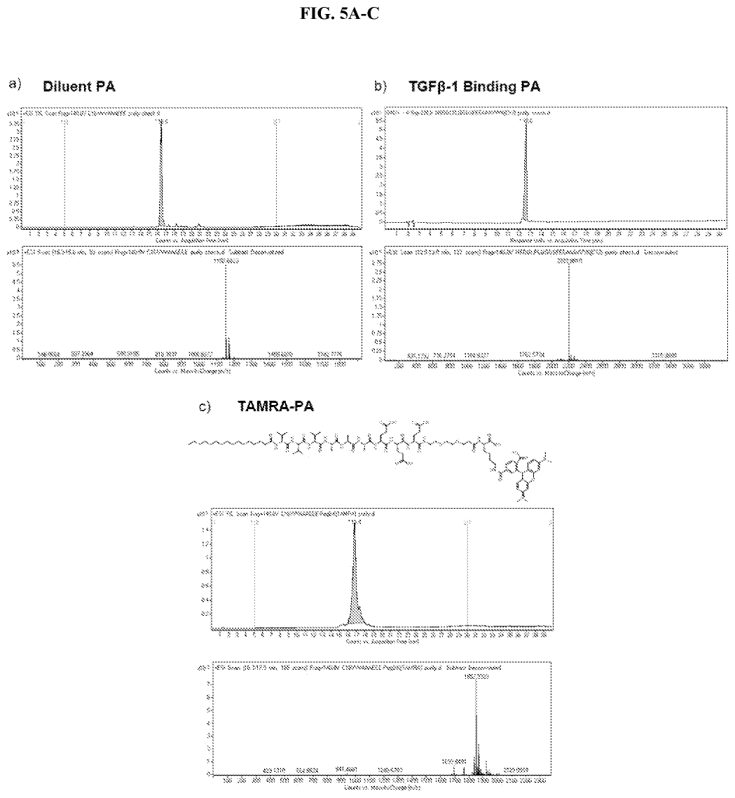

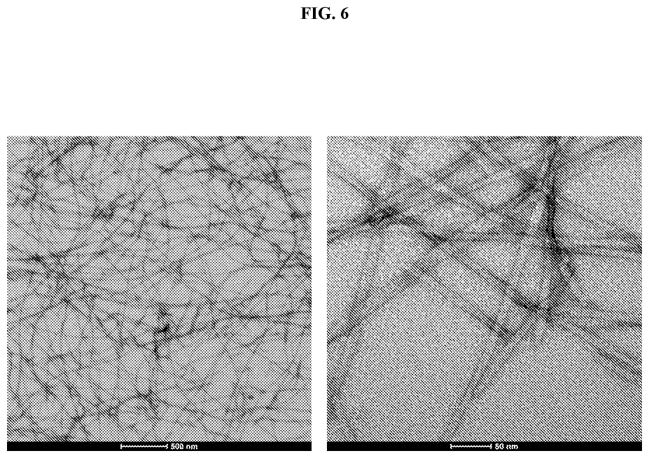

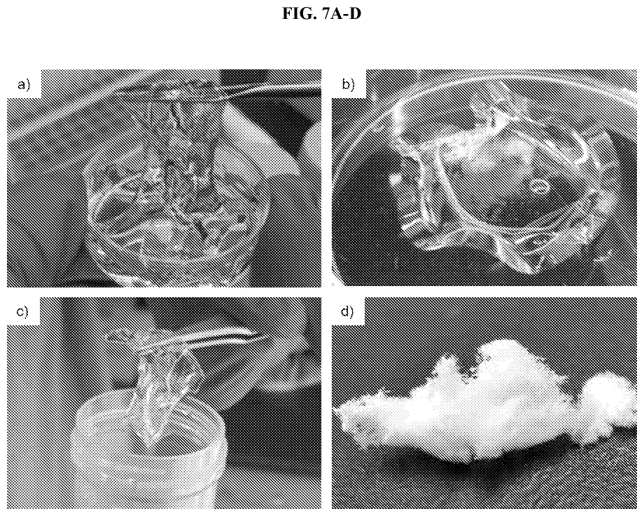

The design of the supramolecular component of the hybrid system was designed based on TGFβ-1 binding filaments, which include a bioactive PA displaying a TGFβ-1 binding sequence and a diluent PA without a bioactive sequence (Ref. 23; incorporated by reference in its entirety) (FIG. 1a; FIG. 5a,b). The binding sequence contained an asparagine-to-aspartic acid mutation based results showing improved growth factor retention by the mutated sequence when displayed on the surface of a PA filament (Ref. 40; incorporated by reference in its entirety). This molecule was coassembled at a 1:9 ratio with an anionic diluent PA known to form gels following charge screening by divalent cations (Ref. 31; incorporated by reference in its entirety). Transmission electron microscopy confirmed the coassemblies formed filaments following heat treatment at 80° C. (FIG. 6). Crosslinked hydrogel microparticles were shown to produce porous, injectable materials with voids between the particles facilitating cell infiltration (Ref. 41, incorporated by reference in its entirety). HA was cross-linked with l-lysine methyl ester crosslinker and N-(3-Dimethylaminopropyl)-N′-ethylcarbodiimide hydrochloride as the coupling agent prior use to further improve its mechanical properties These gels were shredded and dried to provide a porous architecture for cell infiltration (FIG. 7). The extent of crosslinking in the HA microgels was determined to be 21±3% by comparing the change in the elemental composition of the gels which determined the amount of the crosslinker incorporated to the number of primary amine groups remaining as a result of incomplete crosslinking as quantified using a colorimetric ninhydrin assay (Table 3).

TABLE 3
Nitrogen content of unmodified and crosslinked HA determined by elemental
analysis with the amount of incorporated crosslinker necessary for the given
increase in nitrogen; and primary amine content of unmodified and crosslinked
HA as determined by ninhydrin assay with fraction of amine modified HA monomers.
Crosslinking extent calculated by subtracting fraction of free amine modified
from total incorporated crosslinker molecules to give total crosslinker
with both amine groups binding HA polymer chains.

| | | | Fold Increase | |
|---|---|---|---|---|
| | % Nitrogen | | Nitrogen | Crosslinker/HA (%) |
| | Unmodified HA | Crosslinked HA | Crosslinked HA | Crosslinked HA |
| | 3.13 | 4.89 | 1.57 | 36 |
| | 3.09 | 5.04 | 1.62 | 39 |
| | 3.11 | 4.95 | 1.59 | 37 |
| Mean | 3.11 | 4.96 | 1.59 | 38 |
| STD | 0.02 | 0.08 | 0.03 | 2 |

Molecular weight of crosslinked HA = 477.2 mg/mmol HA monomer

| | Primary Amine Groups (μmol/mg) | | Primary Amine/HA (%) |
|---|---|---|---|
| | Unmodified HA | Crosslinked HA | Crosslinked HA |
| | 0.03 | 0.37 | 18 |
| | 0.03 | 0.33 | 16 |
| | 0.02 | 0.35 | 17 |
| | 0.04 | 0.34 | 16 |
| | 0.02 | 0.43 | 20 |
| Average | 0.03 | 0.36 | 17 |
| STD | 0.01 | 0.04 | 2 |

Extent of crosslinking = 21 ± 3%

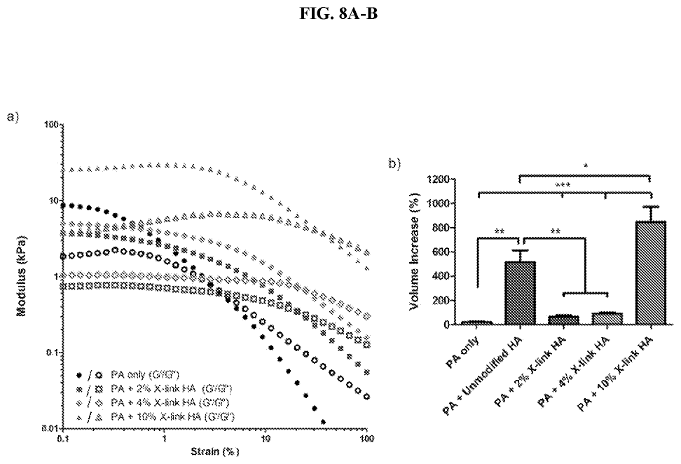

To produce the hybrid material, a solution of PA filaments was added to the lyophilized HA particles and stirred with a spatula. After 1 h the phases combined to form a think putty that could be formed into a desired shape, yet could still be ejected through a syringe. Qualitatively, it was found that combining PA solutions prepared at 2 wt % added to HA particles for a final concentration of 4% HA had the best handling properties for use in surgery. To assess the change in performance of the material due to the addition of crosslinked HA, rheological strain sweeps were performed on the material with ionic CaCl₂) added to stiffen the PA component of the hybrid. The addition of HA significantly improved the flow strain of the material, indicating the HA based material would be more resilient to shear strain in vivo (FIG. 1b). While the flow strain was higher for PA combined with unmodified HA, PA/HA gels made with crosslinked particles were significantly stiffer at low strain than the soft gels made from unmodified HA (5.0±1.0 kPa vs. 1.1±0.2 kPa). Crosslinking the HA particles significantly decreased swelling of the hybrid, indicating that gels made with crosslinked HA would be better maintained in cartilage defects than gels made with unmodified HA (FIG. 1c). Degradation of the HA component of the material was also measured to ensure the implant would not be cleared too quickly from the defect site. HA without added PA degraded completely within 6 d when incubated in a hyaluronidase solution, but when prepared as a hybrid with PA the degradation of HA slowed significantly, with the slowest rate in gels prepared with PA and crosslinked HA (FIG. 1d). To validate the PA/HA hybrid with 4% crosslinked HA, we compared rheological properties and swelling at several crosslinked HA concentrations. These experiments showed the 4% crosslinked HA composition had similar flow strain and swelling properties and a higher storage modulus at low strain compared to 2% crosslinked HA compositions, while 10% crosslinked HA compositions swelled excessively (FIG. 8). Together, these data supported use of the PA/HA hybrid with cross-linked HA particles in large-animal studies based on their increased toughness, low swelling, and slow degradation properties.

### PA HA Hybrid Implantation and Retention

Retention of the mechanically improved PA/HA hybrid material was assessed in osteochondral defects made in a large animal model to determine if the material could remain present long enough to effect cartilage healing. An ovine model was selected for in vivo studies due to the similarities in weight bearing and in anatomy of sheep hind-stifles and to human knees (Refs. 42-43; incorporated by reference in their entireties). Osteochondral defects were made in both the weight-bearing medial femoral condyle and in the non-weight-bearing femoral trochlear groove, to determine how mechanical shear stress affects the materials performance. At each location, the defects were critically sized at 7 mm wide (Ref. 44; incorporated by reference in its entirety) and were 3-4 mm deep. Although human knee cartilage can exceed 3 mm in thickness (Ref. 45; incorporated by reference in its entirety), the cartilage in ovine stifles is around 0.5 mm thick (Ref. 46; incorporated by reference in its entirety), meaning the defects extended into the subchondral bone and induced bleeding. Defects were filled by ejecting the PA/HA hybrid from a syringe fitted with an 18-gauge needle and packing the material to fill the defect space (FIG. 1e). The ability to form the implant into the shape of the defect was crucial, as even custom fabricated implants often fail to integrate when shaped prior to implantation (Ref. 47; incorporated by reference in its entirety). A calcium chloride solution was then dripped on the defect in order to induce gelation of the PA filaments, stiffening the implant surface.

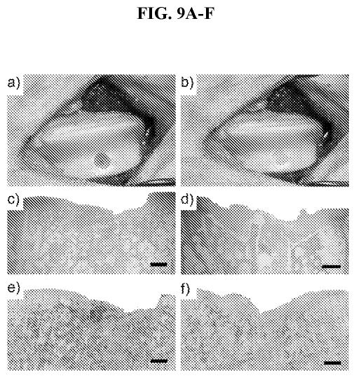

To assess material retention, defects in both stifles were filled with a hybrid gel containing TAMRA-tagged PA filaments (FIG. 1f,g; FIG. 5c; FIG. 9a,b), and one hindlimb was placed in a sling following surgery while the other was loadbearing. After 7 d, hematoxylin and eosin (H&E) staining showed good filling of both condyle defects and trochlea defects in both the slung and non-slung stifles. Fluorescent imaging of unstained sections showed the fluorophore remained throughout the defects, confirming material filling the defect retained the bioactive PA filaments (FIG. 1h-k; FIG. 9c-f). Retention of the bioactive component of the implant material for 7 d indicated the material could withstand the mechanical shear stress present in large-animal joints and influence healing in longer term experiments, even in fully load-bearing joint surfaces without the use of a sling.

### Self-Assembly of PA HA Hybrid Material

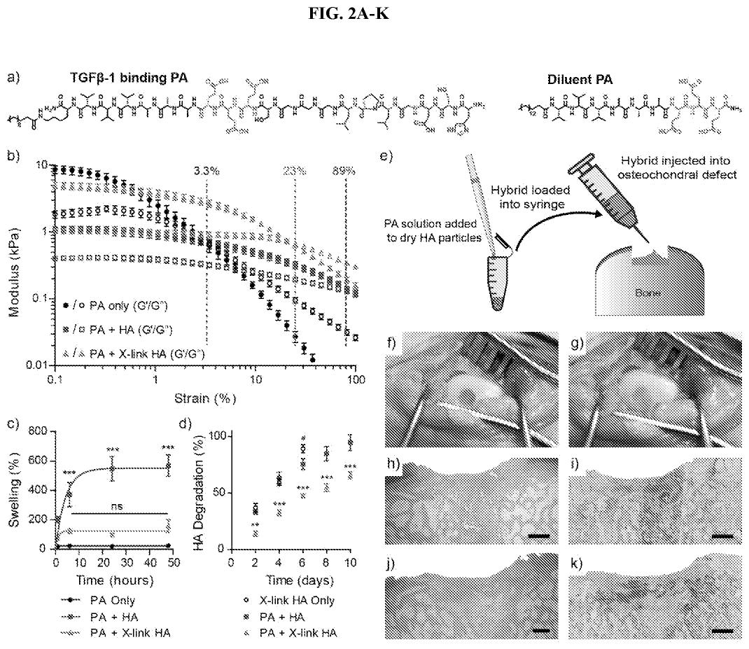

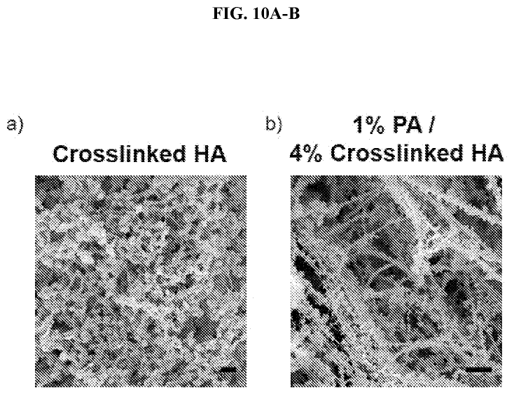

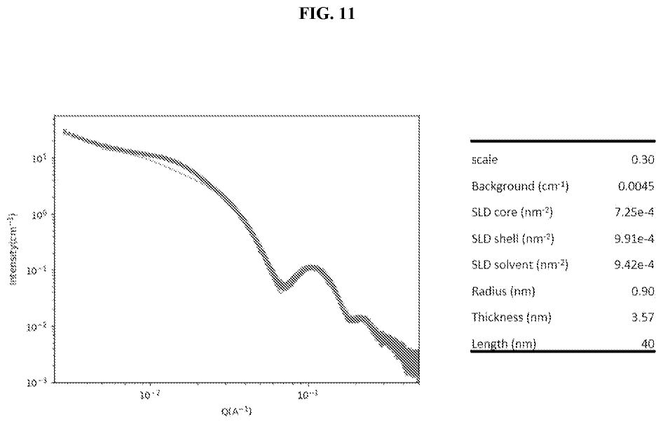

Having established that the PA/HA hybrid material had the mechanical properties necessary to fill large animal cartilage defects, experiments were conducted during development of embodiments herein to determine how combining the PA and HA components affected the self-assembly of the system. Scanning electron microscopy (SEM) revealed that while PA alone formed a dense network of randomly oriented filaments, the PA/HA hybrid material comprised bundles of filaments with large pores between the bundles. These bundles were not HA particles alone, which appeared as sphere-like particles when dehydrated for SEM FIG. 10a). Rather, bundles formed with the addition of 2-4% HA, but not with the addition of 10% HA to the PA solution (FIG. 2a,b; FIG. 10b). The morphology and molecular organization of these structures was characterized using a simultaneously collected small-angle X-ray scattering (SAXS), mid-angle scattering (MAXS), and wide-angle X-ray scattering (WAXS) patterns. The SAXS pattern produced by PA alone in solution fit a core-shell cylinder model with a fiber diameter of 8 nm (FIG. 11). In the WAXS region, Bragg peaks with associated d-spacings of 0.47 nm and 0.24 nm were consistent with β-sheet formation among the PA molecules. In the MAXS region, a peak corresponding to a d-spacing of 0.99 nm was observed which can be attributed to the spacing between β-strands within the filaments. In the PA/HA hybrid material, a strong Bragg peak at a d-spacing of 15 nm appeared, which was a result of regular spacing of the filaments within the observed aggregates. This spacing was less than twice the diameter of each fiber, indicating that the filaments within the superstructures were packed close to one another. In these patterns, the characteristic form factor for cylindrical fibers remained and the position of the higher-q Bragg peaks was unchanged, suggesting the fiber structure did not change with bundling. The low-q Bragg peak was significantly weakened with 10% added HA, consistent with the decreased bundling of filaments in this sample observed by SEM (FIG. 2c).

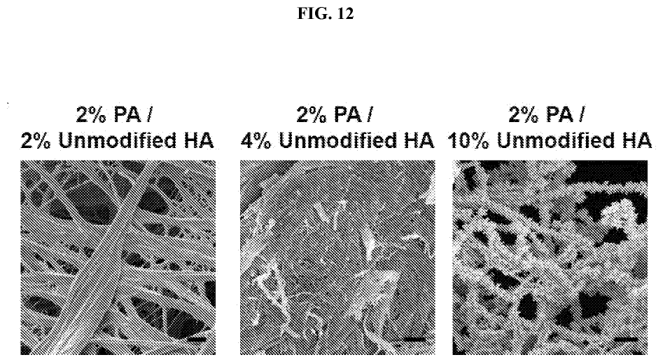

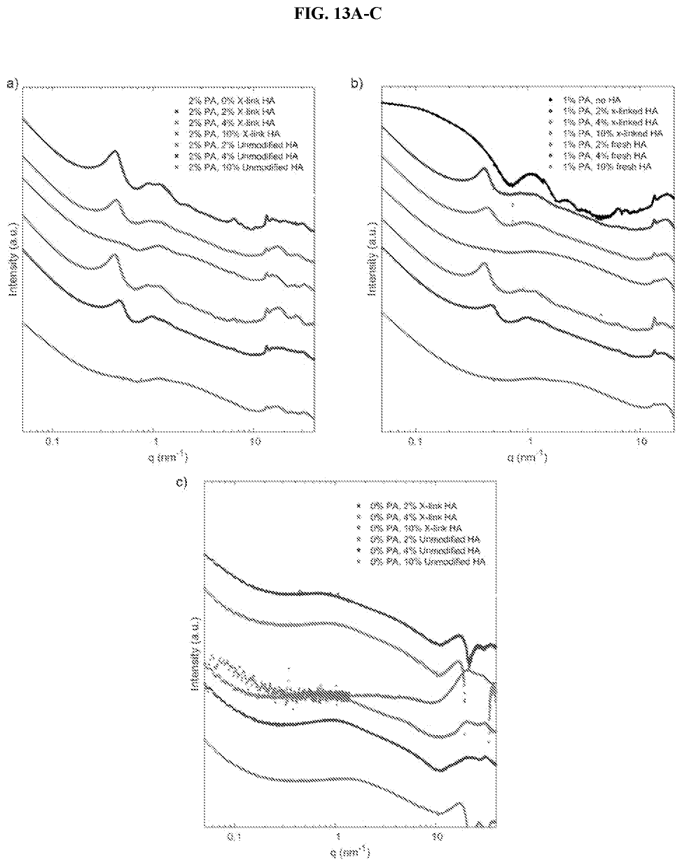

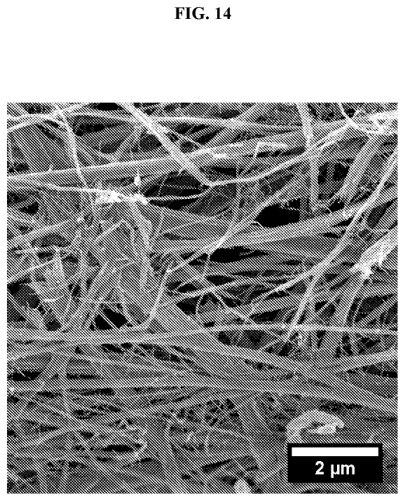

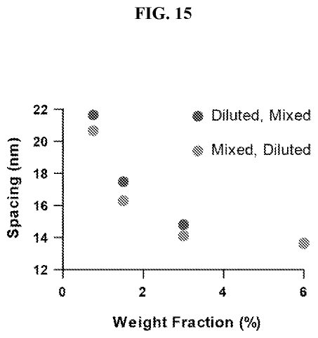

The mechanism of bundle formation in the hybrid system was investigated. The observed bundling was not a result of HA crosslinking (or amine groups remaining from incomplete crosslinks) as SEM showed similar filament aggregates FIG. 12) and SAXS showed a similar Bragg peak FIG. 13) for the PA/HA hybrid made with unmodified HA at the same concentrations. In addition, bundling was not due to interactions between the positively charged binding epitope and the negatively charged HA, as bundles were observed when diluent PA only filaments were added to HA particles FIG. 14). These results led to the hypothesis that bundle formation was a result of the high hydrophilicity of HA. X-ray induced ionization of PA filaments causes fiber alignment to maximize the distance between the highly charged fibers (Ref. 48; incorporated by reference in its entirety). In the hybrid system, PA filaments align due to HA's higher propensity for osmotic swelling relative to PA filaments. When the two components are combined, the available water is primarily associated with swollen HA particles, confining the PA filaments to a limited volume. In this confined space, fibers aligned to maximize distance between their negatively charged surfaces, indicating that at a spacing of 15 nm the osmotic force produced by the swelling HA equaled the repulsive force between neighboring filaments. PA and HA were combined at the normal ratio and diluted with water by physical mixing to change the weight fraction of the material. Following dilution, inter-filament distance increased as a function of decreasing concentration, indicating that the regular spacing of PA filaments was a result of volume confinement and not a result of attractive interactions among the assemblies (FIG. 15). Thus, the microstructure of the hybrid material comprises dense PA bundles made of peptides surrounded by a highly solvated glycosaminoglycan matrix (FIG. 2b). This architecture mimics the organization of hyaline cartilage were fibrous collagen is surrounded by a web of solvated glycosaminoglycans including HA and CS (ref. 49; incorporated by reference in its entirety).

### Chondrogenesis in PA HA Hybrid Gels

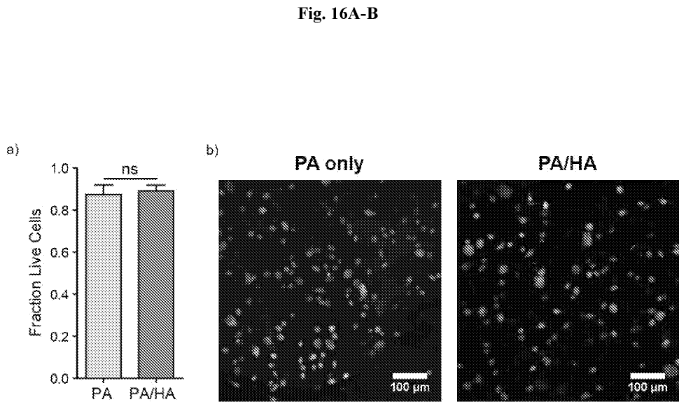

The chondrogenic potential of cells encapsulated in the cartilage mimetic scaffolds was confirmed to ensure that the addition of HA particles did not inhibit the bioactivity of the PA filaments. It was demonstrated that cells could survive encapsulation by mixing human mesenchymal stem cells (hMSCs) in suspension with a solution of PA fibers before adding the combination to dried crosslinked HA particles and gelling with ionic calcium. A live/dead assay showed 89±2% of cells were viable after 24 h, which was statistically similar to survival following encapsulation in PA only gels FIG. 16). The morphology of the encapsulated cells was investigated by confocal microscopy and by SEM. Fluorescent microscopy showed rounded cells dispersed within both PA only gels and hybrid gels (FIG. 2d,e). SEM showed cells in cavities between the bundles, with cell extensions binding to filament bundles (FIG. 2f). It was confirmed that PA nanofibers retained their bioactivity with the addition of HA by quantifying growth factor release from the hybrid material. Compared to hybrid gels made with diluent PA only, hybrid gels that included PA filaments presenting the TGFβ-1 binding epitope retained more growth factor over 3 weeks, although a significant majority of the growth factor remained in both formulations during the experiment (FIG. 2g). The high retention by both gels is likely due to the tight bundling of PA nanofibers, limiting diffusion out of the bundles. After 4 weeks of culture, the chondrogenic potential of the gels was quantified by measuring production of sulfated GAG (sGAG) for both PA only and PA/HA gels in serum free chondrogenic media without added growth factor, with 10 ng/ml TGFβ-1 supplemented media, and with TGFβ-1 encapsulated in the gels. The experiment was designed so that over 4 weeks the total amount of TGFβ-1 added in the media supplemented group from aliquots freshly thawed at each media change equaled the total amount of TGFβ-1 encapsulated in the gels at the start of the experiment. For both PA only and PA/HA gels, the addition of TGFβ-1 either in the media throughout the experiment or in the gels at the start improved normalized sGAG content (FIG. 2h). For PA only gels, there was no statistical difference between gel encapsulation or media supplementation, but in hybrid gels encapsulation induced significantly more normalized sGAG production than continuous media supplementation with TGFβ-1. Thus, TGFβ-1 encapsulation by the hybrid gels maintained growth factor activity throughout the experiment, inducing chondrogenesis at least as well as periodic addition of freshly thawed growth factor. There was no statistically significant difference in normalized sGAG production for TGFβ-1 encapsulating PA gels relative to PA/HA hybrid gels, indicating the improvements in physical performance did not come at the expense of bioactivity. These experiments verified the chondrogenic potential for the hybrid gel, recommending their use for cartilage regeneration.

### Defect Filling and Integration in Response to Exogenous TGFβ-1 Delivery

To evaluate the ability of the mechanically improved PA/HA hybrid to influence cartilage healing, a short-term study aimed at evaluating defect filling and material integration with host cartilage was performed. In previous work in a rabbit model, TGFβ-1 binding PA filaments improved cartilage healing both with and without the addition of TGFβ-1, presumably due to the ability of the material to bind endogenous growth factor (Ref. 23; incorporated by reference in its entirety). To determine if exogenous growth factor was necessary in the large animal model, sheep were divided into two groups of six sheep. In the first group, (+) TGFβ, the hybrid was loaded with 1 μg/mL TGFβ-1 prior to implantation in a condyle defect and a trochlea defect in one stifle, while an equal concentration of the growth factor in saline was dripped into control defects made in the contralateral stifle. While this dose was greater than the TGFβ-1 dose used in prior rabbit studies (100 ng/mL), this is the lowest TGFß dose reported in a material delivering the TGFβ-1 or TGFβ-3 to a full-thickness cartilage or osteochondral defect in a large-animal model (Refs. 15, 50-52: incorporated by reference in their entireties). In the second group, (−) TGFβ, no TGFβ-1 was added to the material prior to implantation in a condyle defect and a trochlear defect in one stifle and only saline was added to the control defects in the contralateral stifle. Because of high variability in sheep age, size, and condition prior to surgery, the aim of this experiment design was to compare the PA/HA hybrid treated defects with control defects in the same animal population.

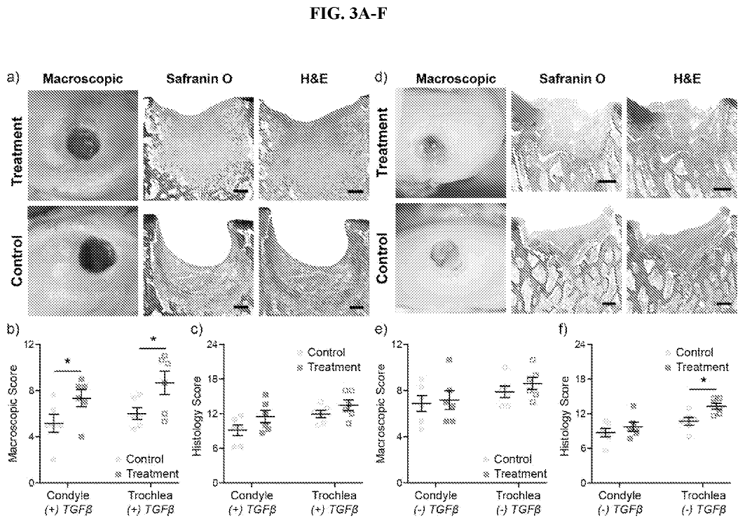

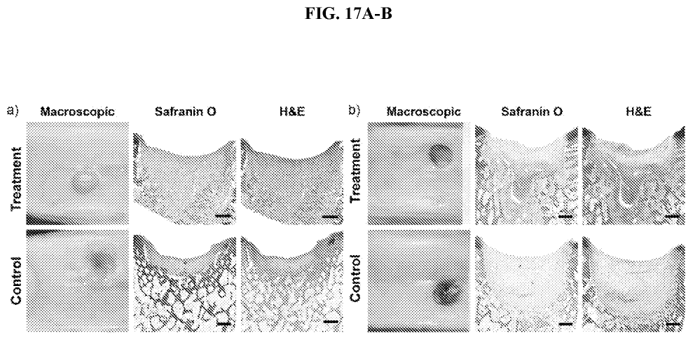

Following surgery, animals were allowed to move freely and were sacrificed after 4 weeks. Macroscopic scoring by blinded experts of defect fill, surface, and integration showed a statistically significant (p<0.05) improvement median score in PA/HA hybrid treated condyle defects and trochlear groove defects relative to controls treated with the growth factor alone (FIG. 3a,b; FIG. 17a; Table 2; Table 4). Scoring of histological sections of the condyle defects and of the trochlear groove defects stained with Safranin O to visualize sGAG or with H&E showed a similar trend to macroscopic scoring with higher median scores for hybrid treated defects, though the difference was not statistically significant (FIG. 3c; Table 2; Table 5). Because 4 weeks is an early timepoint in the cartilage regeneration process, it was expected that the macroscopic appearance would be more indicative of material response than staining for markers of hyaline tissue which develop at later times. Still, in the PA/HA hybrid treated defects of the (+) TGFβ group showed instances of strong integration between the defect fill and native tissue, indicating strong compatibility of the implant material with surrounding tissue. In the (−) TGFβ group, there was no statistical difference in macroscopic scores for defects treated with the material alone relative to defects filled with the saline vehicle only, though histological scoring indicated improvement in trochlear groove defects relative to untreated controls (FIG. 3d-f;

FIG. 17b; Table 2). While the average macroscopic score of all treated and untreated condyle defects was higher for the (−) TGFβ group than for the (+) TGFβ group, this difference was attribute to animal-to-animal variability between the small populations included in each group. Improved macroscopic scores due to TGFβ-1 loaded PA/HA hybrid implants indicated the supramolecular scaffold had the physical integrity needed to support improved filling and integration of cartilage defects, both in in the trochlear groove and in the more mechanically active femoral condyle.

TABLE 2
Comparison of median macroscopic scores (12-point scale) and median histology
scores (24-point scale) of PA/HA treated and control defects in 4-week and 12-week sample
groups with number of animals in each group and p-value calculated using a non-parametric
Man-Whitney test.

| | | | Macroscopic Score | | | | Histology Score | | | |
|---|---|---|---|---|---|---|---|---|---|---|
| | | TGFβ | Treated | Control | n | p | Treated | Control | n | p |
| Condyle | 4 weeks | (+) | 7.83 | 5 | 6 | 0.045 | 13.7 | 9.7 | 6 | 0.11 |
| | 4 weeks | (−) | 6.83 | 7 | 6 | 0.5 | 9.2 | 8.8 | 6 | 0.34 |
| | 12 weeks | (+) | 9.33 | 7 | 13 | 0.064 | 10.67 | 10 | 13 | 0.36 |
| | 24 weeks | (+) | 6.8 | 9 | 2 | | 20.3 | 15.3 | 2 | |
| Trochlea | 4 weeks | (+) | 9.5 | 5.7 | 6 | 0.033 | 13.7 | 10.3 | 6 | 0.15 |
| | 4 weeks | (−) | 8.5 | 7.8 | 6 | 0.19 | 13.3 | 10.8 | 6 | 0.008 |
| | 12 weeks | (+) | 10 | 10.33 | 13 | 0.41 | 13.3 | 11 | 13 | 0.042 |

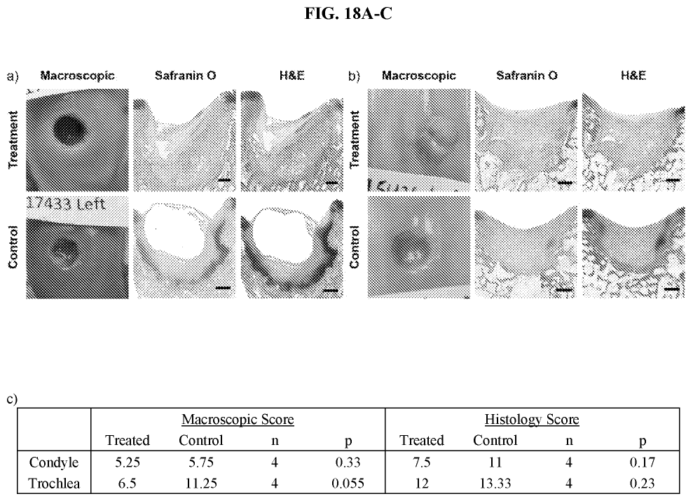

Because only the TGFβ-1 treated group showed improved macroscopic scores at 4 weeks, it was investigated whether the material without exogenous TGFβ-1 loading would be able to improve macroscopic or histological appearance at later times. A 4-animal pilot study was implemented in which (−) TGFβ group sheep were sacrificed after 12 weeks. Evaluation of cartilage appearance showed application of the material without the addition of the growth factor did not improve the macroscopic appearance or histological scores for defects in either the condyle or the trochlea at this later time (FIG. 18). The lack of score improvement in the PA/HA hybrid treated defects without exogenous TGFβ-1 may be related to decreased physiological growth factor availability in large animals like sheep relative to smaller mammals like rabbits. Alternatively, the tight bundles of PA filaments that formed following the addition of HA may have limited the ability of the displayed binding epitope to bind the growth factor after implantation by inhibiting diffusion of endogenous growth factor between PA filaments. Though these results indicated exogenous growth factor may in some situations be needed for cartilage repair, the improvement observed at 4 weeks in the (+) TGFβ group suggested the PA/HA hybrid material could facilitate cartilage repair with an unprecedentedly small dose of exogenous growth factor.

### Effect of PA HA Hybrid on Cartilage Healing

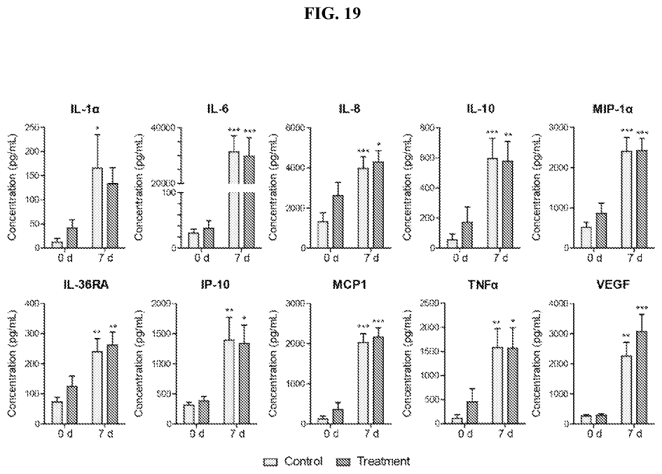

Based on the improved macroscopic scores observed in both condyle and trochlear defects in short-term studies, on (+) TGFβ scaffolds were focused on to evaluate cartilage regeneration at 12 weeks. 12 weeks was chosen expecting that this would be too early for hyaline-like tissue to develop without intervention, but long enough that cartilage repair could occur in response to the bioactive scaffold. For these sheep, joint fluid samples were drawn prior to surgery and 1 week following implantation of the material. Biomarker concentration in the joint fluid was quantified by a multiplexed immunoassay to better understand how defect formation and material implantation affect the biology of the joint space. Fourteen sheep were initially included in this study, though evaluation of the joint fluid drawn prior to surgery showed significantly elevated tumor necrosis factor-α (TNF-α) in one of its joints, giving a concentration relative to the contralateral joint over three standard deviations larger than the mean across all animals tested prior to surgery. Because this cytokine is highly correlated with acute inflammation (Ref. 53; incorporated by reference in its entirety), it was determined that this sheep had an underlying condition prior to surgery and excluded the animal from further analysis. In the remaining sheep, concentrations of the ten inflammatory biomarkers tested were significantly elevated 1 week following surgery relative to pre-surgery levels FIG. 19). These included interleukin 6 (joint fluid concentrations of which increased by more than two orders of magnitude following defect formation), interleukin 10, and monocyte chemoattractant protein 1—each an important mediator in the cartilage repair process (Refs. 54-56; incorporated by reference in their entireties). There were no significant differences in inflammatory biomarker connections between treated and control joints, indicating that the PA/HA hybrid does not promote a harmful inflammatory response.

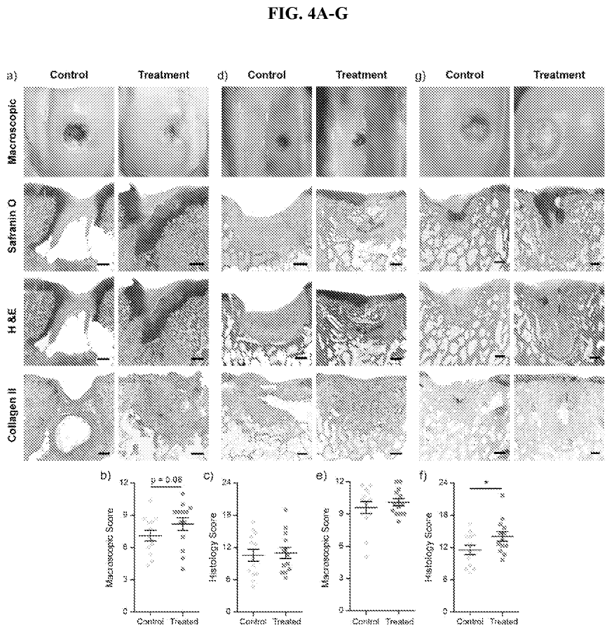

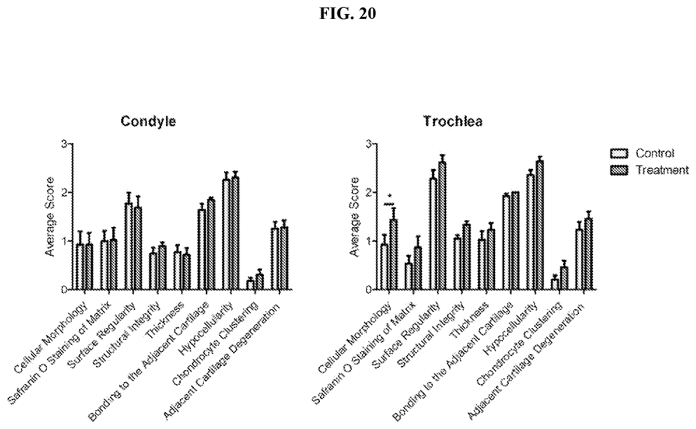

Evaluation of the macroscopic appearance of defects following resection showed a trend of improved scores for condyle defects treated with the hybrid material relative to control defects treated with the growth factor only (FIG. 4a-c; Table 2). In the trochlea, both treated and control defects healed significantly, making it difficult to discern differences in macroscopic appearance between treated and control groups (FIG. 4d-f; Table 2). Histological sections of condyle defects stained with Safranin O or H&E showed high variability in condyle defects with hyaline-like tissue observed in some treated defects, but poor healing in others, resulting in no significant difference in their score (FIG. 4a,c; Table 2). In the trochlear groove, however, treatment with the hybrid material resulted in better repair of hyaline-like tissue overall as indicated by the development of a neocartilage layer rich in sGAG and collagen II (FIG. 4d), resulting in a statistically significant improvement in blinded scores of the histological sections (FIG. 4f; Table 2). Treatment resulted in higher mean scores across all evaluation categories, with the most significant improvement in the development of hyaline-like cellular morphology FIG. 20; Table 5). The difference in response between the condyle defects and the trochlear groove may have been related to differences in healing time course for the two defect sites. In the more mechanically active condyle, slow cartilage healing may have resulted in both a sustained difference in defect fill between treated and control groups for 12 weeks and in limited formation of hyaline-like tissue at that time. On the other hand, faster healing in the trochlear groove may have led to high macroscopic scores regardless of treatment, but enhanced histological scoring only in response to the bioactive hybrid material treatment. To test this hypothesis, bilateral osteochondral defects were made in the condyles of two additional sheep and evaluated 24 weeks post-surgery. At this later time, representative histological sections showed cartilage-like tissue positive for collagen II covered the defect surface flush with the surrounding tissue (FIG. 4g), leading to an improved median histological score relative to the untreated control defects. Although this group included only two animals, these results reveal that PA/HA hybrid treatment can lead to improved regeneration of hyaline cartilage in weight bearing femoral condyle defects.

The efficacy of the PA/HA hybrid material in these 12-week and 24-week studies is especially surprising when considered in context of the experimental limitations of the present large-animal study. This study used sheep acquired from university research herds with a wide range of ages and weights, which likely contributed to the wide variability of macroscopic and histological scores within the treatment and control groups. To address this variability, experiments were conducted during development of embodiments herein to to create bilateral defects and treat only one side, which ensured treatment and control defects were from the same sheep population in each study group. Still, differences between joints may develop within the same animal due to inflammation or anatomical changes that can occur with age. Importantly, both 4-week and 12-week studies demonstrated statistically significant differences in macroscopic and histological scores, respectively, suggesting a strong effect that could be measured despite these confounding factors. In addition, the use of bilateral defects may have limited the efficacy of the implanted material by affecting the behavior of the sheep following surgery. Although the 1-week pilot study showed little difference in defect fill between the slung limb and contralateral unslung limb, placing one limb in a sling likely decreased the sheep's movement following surgery. It was noted that when no limbs were placed in slings for the 4-week and 12-week studies, sheep were more active in the days following surgery, which may have resulted in higher mechanical shear on the defect relative to the non-slung limb in the pilot study. This effect may have contributed to the more irregular surface morphology observed in histological sections of treated condyle defects relative to treated trochlear groove defects. Therefore, it is expected that if the movement of the sheep was restricted following surgery, improvement due to the PA/HA hybrid implant would have been further increased. This is important because unlike the sheep in this study, the knees of clinical patients are immobilized for some time following cartilage repair treatments.

Experiments were conducted during development of embodiments herein (e.g., throughout Example 2) to develop an injectable bioactive cell-free scaffold capable of inducing cartilage regeneration in a large-animal sheep model. By combining a bioactive-self assembling component with HA microgels, a resilient material was produced with the mechanical properties necessary to improve osteochondral defect fill over short times and the biological properties necessary to induce chondrogenesis in response to sustained delivery of a low TGFβ-1 dose at longer times. Characterization of the material revealed a previously unknown phenomenon whereby combining the supramolecular PA and covalent HA components produced bundled, porous scaffolds. These biocompatible gels supported chondrogenesis of embedded progenitor cells and may be useful in other regenerative applications where scaffold topology is important in cellular response. Due to the chemical versatility of the supramolecular PA fibers, the system described herein can be adapted to deliver additional growth factor binding or signaling peptides. Based on this work, the PA/HA hybrid provides a materials platform that can be delivered arthroscopically, withstand the mechanical stresses of large-animal joints, and display programmed bioactive signals tailored to any number of bioregenerative applications. The ability of the hybrid gel to induce repair of hyaline cartilage in the mechanically active large-animal condyle surface indicate the strong potential of the material to improve cartilage repair in human patients.

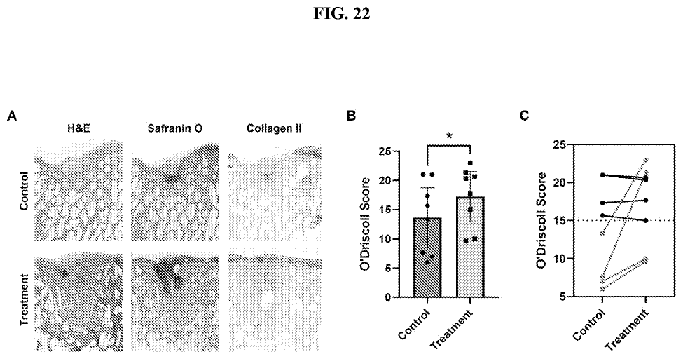

Based on the material retention and chondrogenesis results at early time points, experiments were conducted during development of embodiments herein to test additional sheep using the weight bearing condyle defect model at the clinically relevant end point of 6 months post operative. Each sheep received identical critical sized defects (˜7 mm diameter, ˜4-5 mm deep) in the medial condyle of each stifle with the control side defect filled with 1 μg/mL TGFβ-1 in saline and the contralateral treatment side defect filled with 1 μg/mL TGFβ-1 encapsulated in the PA/HA slurry. Robust glycosaminoglycan and collagen II deposition was observed in the histological sections of condyle defects treated with the PA/HA slurry, indicating robust hyaline cartilage formation (FIG. 22A). Compared to control defects in the same animal, statistically significant improvement in O'Driscoll scoring of the histological sections was observed for the PA/HA treated defects, demonstrating the efficacy of the composite material for long term cartilage repair in a large animal model (FIG. 22B). For animals where the control side defect exhibited low O'Driscoll scores (the four lowest scoring animals were all below 15), their treatment side defects had the largest improvement in histological scoring (FIG. 22C, red points). In comparison, animals with O'Driscoll scores above 15 in the control side did not show improvement in the treatment side, likely due to a ceiling effect of the bounded scoring system (i.e., these animals had an inherently high healing capacity so there was not room for improvement). Taken together, this data indicates that the PA/HA slurry is particularly useful for challenging cartilage defects or patients with poor cartilage healing capacity.

### REFERENCES

The following references, some of which are cited above by number, are herein incorporated by reference in their entireties.
1. D. J. Huey, J. C. Hu, K. A. Athanasiou, Unlike bone, cartilage regeneration remains elusive. Science 338, 917-921 (2012).
2. J. Malda, J. Groll, P. R. van Weeren, Rethinking articular cartilage regeneration based on a 250-year-old statement. Nature Reviews Rheumatology 15, 571-572 (2019).
3. A. J. Price et al., Knee replacement. The Lancet 392, 1672-1682 (2018).
4. A. R. Armiento, M. J. Stoddart, M. Alini, D. Eglin, Biomaterials for articular cartilage tissue engineering: Learning from biology. Acta Biomaterialia 65, 1-20 (2018).
5. J. R. Steadman et al., Outcomes of microfracture for traumatic chondral defects of the knee: average 11-year follow-up. Arthroscopy 19, 477-484 (2003).
6. M. L. Redondo, A. J. Beer, A. B. Yanke, Cartilage restoration: Microfracture and osteochondral autograft transplantation. Journal of Knee Surgery 31, 231-238 (2018).
7. D. K. Bae, K. H. Yoon, S. J. Song, Cartilage healing after microfracture in osteoarthritic knees. Arthroscopy 22, 367-374 (2006).
8. P. C. Kreuz et al., Results after microfracture of full-thickness chondral defects in different compartments in the knee. Osteoarthritis Cartilage 14, 1119-1125 (2006).
9. L. Andriolo et al., Cell-free scaffolds in cartilage knee surgery: A systematic review and meta-analysis of clinical evidence. Cartilage 10.1177/1947603519852406, 1-16 (2019).
10. N. Eslahi, M. Abdorahim, A. Simchi, Smart polymeric hydrogels for cartilage tissue engineering: A review on the chemistry and biological functions. Biomacromolecules 17, 3441-3463 (2016).
11. L. Bian, M. Guvendiren, R. L. Mauck, J. A. Burdick, Hydrogels that mimic developmentally relevant matrix and N-cadherin interactions enhance MSC chondrogenesis. Proceedings of the National Academy of Sciences of the United States of America 110, 10117-10122 (2013).
12. D. A. Wang et al., Multifunctional chondroitin sulphate for cartilage tissue-biomaterial integration. Nature Materials 6, 385-392 (2007).
13. B. Sharma et al., Human cartilage repair with a photoreactive adhesive-hydrogel composite. Science Translational Medicine 5, 167ra166 (2013).
14. T. L. Spain, C. M. Agrawal, K. A. Athanasiou, New technique to extend the useful life of a biodegradable cartilage implant. Tissue Engineering 4, 343-352 (1998).
15. I. L. Kim et al., Fibrous scaffolds with varied fiber chemistry and growth factor delivery promote repair in a porcine cartilage Defect Model. Tissue Engineering Part A 21, 2680-2690 (2015).
16. M. Liu et al., Injectable hydrogels for cartilage and bone tissue engineering. Bone Research 5, 17014 (2017).
17. D. Yang et al., The immune reaction and degradation fate of scaffold in cartilage/bone tissue engineering. Materials Science & Engineering, C: Materials for Biological Applications 104, 109927 (2019).
18. E. B. Hunziker, Growth-factor-induced healing of partial-thickness defects in adult articular cartilage. Osteoarthritis and Cartilage 9, 22-32 (2001).
19. H. Madry, A. Rey-Rico, J. K. Venkatesan, B. Johnstone, M. Cucchiarini, Transforming growth factor Beta-releasing scaffolds for cartilage tissue engineering. Tissue Engineering Part B: Reviews 20, 106-125 (2014).
20. F. Zhou et al., Tough hydrogel with enhanced tissue integration and in situ forming capability for osteochondral defect repair. Applied Materials Today 13, 32-44 (2018).
21. P. M. van der Kraan, The changing role of TGFbeta in healthy, ageing and osteoarthritic joints. Nature Reviews Rheumatology 13, 155-163 (2017).
22. C. Deng, J. Chang, C. Wu, Bioactive scaffolds for osteochondral regeneration. Journal of Orthopaedic Translation 10.1016/j.jot.2018.11.006 (2018).
23 R. N. Shah et al., Supramolecular design of self-assembling nanofibers for cartilage regeneration. Proceedings of the National Academy of Sciences of the United States of America 107, 3293-3298 (2010).
24. J. D. Hartgerink, E. Beniash, S. I. Stupp, Self-assembly and mineralization of peptide-amphiphile nanofibers. Science 294, 1684-1688 (2001).
25. J. D. Hartgerink, E. Beniash, S. I. Stupp, Peptide-amphiphile nanofibers: a versatile scaffold for the preparation of self-assembling materials. Proceedings of the National Academy of Sciences of the United States of America 99, 5133-5138 (2002).
26. G. A. Silva et al., Selective differentiation of neural progenitor cells by high-epitope density nanofibers. Science 303, 1352-1355 (2004).
27. S. S. Lee et al., Sulfated glycopeptide nanostructures for multipotent protein activation. Nature Nanotechnology 12, 821-829 (2017).
28. E. Sleep et al., Injectable biomimetic liquid crystalline scaffolds enhance muscle stem cell transplantation. Proceedings of the National Academy of Sciences of the United States of America 114, E7919-E7928 (2017).
29 A. N. Edelbrock et al., Supramolecular nanostructure activates TrkB receptor signaling of neuronal cells by mimicking brain-derived neurotrophic factor. Nano Letters 18, 6237-6247 (2018).
30 K. L. Niece et al., Modification of gelation kinetics in bioactive peptide amphiphiles. Biomaterials 29, 4501-4509 (2008).
31. S. Zhang et al., A self-assembly pathway to aligned monodomain gels. Nature Materials 9, 594-601 (2010).
32. R. Freeman, J. Boekhoven, M. B. Dickerson, R. R. Naik, S. I. Stupp, Biopolymers and supramolecular polymers as biomaterials for biomedical applications. MRS Bulletin 40, 1089-1101 (2015).
33. M. A. Greenfield, J. R. Hoffman, M. O. de la Cruz, S. I. Stupp, Tunable mechanics of peptide nanofiber gels. Langmuir 26, 3641-3647 (2010).
34. S. S. Lee et al., Bone regeneration with low dose BMP-2 amplified by biomimetic supramolecular nanofibers within collagen scaffolds. Biomaterials 34, 452-459 (2013).
35. Z. Yu et al., Simultaneous covalent and noncovalent hybrid polymerizations. Science 351, 497-502 (2016).
36. S. M. Chin et al., Covalent-supramolecular hybrid polymers as muscle-inspired anisotropic actuators. Nature Communications 9, 2395 (2018).
37. C. Li et al., Supramolecular-covalent hybrid polymers for light-activated mechanical actuation. Nature Materials 10.1038/s41563-020-0707-7 (2020).
38. R. Freeman et al., Reversible self-assembly of superstructured networks. Science 362, 808-813 (2018).
39. G. Lisignoli et al., Cellular and molecular events during chondrogenesis of human mesenchymal stromal cells grown in a three-dimensional hyaluronan based scaffold. Biomaterials 26, 5677-5686 (2005).
40. J. A. Lewis et al., Transforming growth factor β-1 binding by peptide amphiphile hydrogels. ACS Biomaterials Science & Engineering 6, 4551-4560 (2020).
41. A. C. Daly, L. Riley, T. Segura, J. A. Burdick, Hydrogel microparticles for biomedical applications. Nature Reviews Materials 5, 20-43 (2019).
42. Y. Lu, M. D. Markel, C. Swain, L. D. Kaplan, Development of partial thickness articular cartilage injury in an ovine model. Journal of Orthopaedic Research 24, 1974-1982 (2006).
43. C. J. Moran et al., The benefits and limitations of animal models for translational research in cartilage repair. Journal of Experimental Orthopaedics 3, 1 (2016).
44 J. L. Cook et al., Animal models of cartilage repair. Bone & Joint Research 3, 89-94 (2014).
45. R. J. Schmitz, H. M. Wang, D. R. Polprasert, R. A. Kraft, B. G. Pietrosimone, Evaluation of knee cartilage thickness: A comparison between ultrasound and magnetic resonance imaging methods. Knee 24, 217-223 (2017).
46. M. W. Cross, D. D. Frisbie, C. W. McIlwraith, A comparative study of articular cartilage thickness in the stifle of animal species used in human pre-clinical studies compared to articular cartilage thickness in the human knee. Veterinary and Comparative Orthopaedics and Traumatology 19, 142-146 (2018).
47. K. E. Roth et al., Biological responses to individualized small titanium implants for the treatment of focal full-thickness knee cartilage defects in a sheep model. Knee 27, 1078-1092 (2020).
48. H. Cui et al., Spontaneous and x-ray-triggered crystallization at long range in self-assembling filament networks. Science 327, 555-559 (2010).
49 N. P. Cohen, R. J. Foster, V. C. Mow, Composition and dynamics of articular cartilage: structure, function, and maintaining healthy state. Journal of Orthopaedic & Sports Physical Therapy 28, 203-215 (1998).
50. E. Ruvinov, T. Tavor Re′em, F. Witte, S. Cohen, Articular cartilage regeneration using acellular bioactive affinity-binding alginate hydrogel: A 6-month study in a mini-pig model of osteochondral defects. Journal of Orthopaedic Translation 16, 40-52 (2019).
51. M. B. Fisher et al., Cartilage repair and subchondral bone remodeling in response to focal lesions in a mini-pig model: implications for tissue engineering. Tissue Engineering Part A 21, 850-860 (2015).
52. N. Mohan et al., Microsphere-based gradient implants for osteochondral regeneration: a long-term study in sheep. Regenerative Medicine 10, 709-728 (2015).
53. C. Janeway, K. P. Murphy, P. Travers, M. Walport, Janeway's Immunobiology (2008).
54 F. Iannone et al., Interleukin-10 and interleukin-10 receptor in human osteoarthritic and healthy chondrocytes. Clinical & Experimental Rheumatology 19, 139-145 (2001).
55. C. Gabay, Interleukin-6 and chronic inflammation. Arthritis Research & Therapy 8, S3 (2006).
56 A. I. Tsuchida et al., Interleukin-6 is elevated in synovial fluid of patients with focal cartilage defects and stimulates cartilage matrix production in an in vitro regeneration model. Arthritis Research and Therapy 14, R262 (2012).
57 T. Bitter, H. M. Muir, A modified uronic acid carbazole reaction. Analytical Biochemistry 4, 330-334 (1962).
58. R. W. Farndale, C. A. Sayers, A. J. Barrett, A direct spectrophotometric microassay for sulfated glycosaminoglycans in cartilage cultures. Connective Tissue Research 9, 247-248 (1982).
59. M. P. van den Borne et al., International Cartilage Repair Society (ICRS) and Oswestry macroscopic cartilage evaluation scores validated for use in Autologous Chondrocyte Implantation (ACI) and microfracture. Osteoarthritis Cartilage 15, 1397-1402 (2007).
60. S. W. O'Driscoll, F. W. Keeley, R. B. Salter, The chondrogenic potential of free autogenous periosteal grafts for biological resurfacing of major full-thickness defects in joint surfaces under the influence of continuous passive motion. An experimental investigation in the rabbit. Journal of Bone and Joint Surgery 68, 1017-1035 (1986).

1. A composite material comprising: (1) peptide amphiphile (PA) nanofibers, and (2) hyaluronic acid (HA) particles.

2. The composite material of claim 1, wherein the composition comprises a paste-like consistency.

3. The composite material of claim 1, further comprising one or more growth factors.

4. The composite material of claim 1, wherein the HA particles are microparticles or nanoparticles.

5. The composite material of claim 1, wherein the HA particles comprise crosslinked HA.

6. The composite material of claim 5, wherein the HA particles comprise L-lysine methyl ester and 1-Ethyl-3-(3-dimethylaminopropyl) carbodiimide (EDC) crosslinked HA.

7. The composite material of claim 6, wherein the HA particles are 5-50% crosslinked.

8. The composite material of claim 7, wherein the HA particles are 10-30% crosslinked.

9. The composite material of claim 6, wherein the HA particles are about 21% crosslinked.

10. The composite material of claim 1, comprising 2-10% HA.

11. The composite material of claim 10, comprising about 4% HA.

12. The composite material of claim 1, wherein the HA particles comprise oxidized HA.

13. The composite material of claim 12, wherein the HA particles comprise partially-oxidized HA.

14. The composition of one of claims 12 and 13, wherein the HA particles display aldehyde groups suitable for crosslinking to amine groups.

15. The composite material of claim 1, wherein the peptide amphiphile nanofibers are derived from a peptide amphiphile solution.

16. The composite material of claim 1, wherein the peptide amphiphile nanofibers comprise:
(i) a hydrophobic non-peptidic segment;
(ii) a β-sheet-forming peptide segment;
(iii) an acidic peptide segment; and
(iv) a bioactive peptide.

17. The composite material of claim 16, wherein the bioactive peptide binds a growth factor or other bioactive agent.

18. The composite material of claim 17, wherein the epitope peptide binds TGF-β1.

19. The composite material of claim 1, wherein the hydrophobic non-peptidic segment of the bioactive peptide amphiphile comprises an acyl chain.

20. The composite material of claim 19, wherein the acyl chain comprises C₆-C₂₀.

21. The composite material of claim 20, wherein the acyl chain comprises C16.

22. The composite material of claim 1, wherein the β-sheet-forming peptide segment of the bioactive peptide amphiphile comprises a combination of 2-6 V and A residues.

23. The composite material of claim 22, wherein the β-sheet-forming peptide segment of the bioactive peptide amphiphile and the charged peptide amphiphile is selected from VVVAAA, AAAVVV, AAVV, VVAA, AA, VV, VA, or AV.

24. The composite material of claim 1, wherein the acidic peptide segment of the bioactive peptide amphiphile comprises a combination of 1-4 Glu (E) and/or Asp (D) residues.

25. The composite material of claim 24, wherein the acidic peptide segment comprises is selected from E, EE, EEE, D, DD, DDD, ED, DE, EDE, DED, EDD, and DEE.

26. The composite material of claim 1, comprising a backbone PA of the bioactive peptide amphiphile selected from C16-AAEE, C16-AEAE, and C16-VVVAAAEEE.

27. The composite material of claim 16, further comprising a diluent PA comprising:
(i) a hydrophobic non-peptidic segment;
(ii) a β-sheet-forming peptide segment; and
(iii) a charged peptide segment.

28. The composite material of claim 27, wherein the hydrophobic non-peptidic segment of the diluent peptide amphiphile comprises an acyl chain.

29. The composite material of claim 28, wherein the acyl chain comprises C₆-C₂₀.

30. The composite material of claim 29, wherein the acyl chain comprises C16.

31. The composite material of claim 24, wherein the β-sheet-forming peptide segment of the diluent peptide amphiphile comprises a combination of 2-6 V and A residues.

32. The composite material of claim 31, wherein the β-sheet-forming peptide segment of the diluent peptide amphiphile is selected from VVVAAA, AAAVVV, AAVV, VVAA, AA, VV, VA, or AV.

33. The composite material of claim 27, wherein the acidic peptide segment of the diluent peptide amphiphile comprises a combination of 1-4 Glu (E) and/or Asp (D) residues.

34. The composite material of claim 22, wherein the acidic peptide segment of the diluent peptide amphiphile is selected from E, EE, EEE, D, DD, DDD, ED, DE, EDE, DED, EDD, and DEE.

35. The composite material of claim 27, comprising a backbone PA of the diluent peptide amphiphile selected from C16-AAEE, C16-AEAE, and 16-VVVAAAEEE.

36. The composite material of claim 27, having a ratio of between 1:1 and 1:25 bioactive PA to diluent PA.

37. The composite material of claim 36, having a ratio of between 1:5 and 1:15 bioactive PA to diluent PA.

38. The composite material of claim 37, having a ratio of about 1:9 bioactive PA to diluent PA.

39. A method of promoting repair of a cartilage and/or osteochondral defect in a subject comprising administering the composite material of claim 1 to the cartilage and/or osteochondral defect.

40. The method of claim 39, wherein the subject exhibits poor cartilage healing capacity.

41. The method of claim 40, wherein the subject has an O'Driscoll score or modified O'Driscoll score of 15 or lower.

42. A method of preparing a composite material of claim 1 comprising mixing a solution of peptide amphiphiles with HA particles in an aqueous solution.

43. A system comprising:
(a) the composite material of claim 1; and
(b) a medical device for applying the composition to a cartilage and/or osteochondral defect in a subject.

44. The system of claim 43, wherein the medical device is a syringe, catheter, or paste applicator.

45. A system comprising:
(a) the composite material of claim 1; and
(b) TGF-β1.
